(19) United States
(12) Patent Application Publication
(10) Pub. No.: US 2026/0229313 A1
(43) Pub. Date: Aug. 6, 2026

(54) GENERATING SEPARABILITY AND DISJOINTNESS MEASURES FOR PAIRWISE INTERACTION DETECTION AND ACTIVE LEARNING

(71) Applicant: Recursion Pharmaceuticals, Inc., Salt Lake City, UT (US)

(72) Inventors: Jason Siyanda HARTFORD, London (GB); Zuheng XU, Vancouver (CA)

(21) Appl. No.: 19/046,270

(22) Filed: Feb. 5, 2025

(51) Int. Cl. G16B 40/10

(52) U.S. Cl. CPC G16B 40/10

(57) ABSTRACT

The present disclosure relates to systems, non-transitory computer-readable media, and methods that implement a framework for determining measures of biological activity for pairwise interactions. Indeed, in one or more implementations, the disclosed systems generate a first set of individual perturbation representations from a first set of cells exposed to a first perturbation and a second set of individual perturbation representations, from a second set of cells exposed to a second perturbation. For instance, the disclosed systems combine the first individual perturbation representation and the second individual perturbation representation to determine a predicted pairwise representation. Moreover, in some instances, the disclosed systems generate a pairwise representation from a group of cells exposed to both the first and second perturbation. Additionally, from comparing the predicted pairwise representation with the pairwise representation, the disclosed systems generate a measure of biological activity of the first and second perturbation.

### BACKGROUND

Recent years have seen significant developments in hardware and software platforms that utilize machine learning to model and predict the complex underlying interactions of cellular behavior. For example, conventional systems utilize various machine learning approaches to generate biological predictions based on various input signals regarding variations to internal or external cellular environments. Despite these recent advances, conventional systems suffer from a number of technical deficiencies, particularly with regard to efficiency, accuracy and operational inflexibility of machine learning models and implementing computing systems.

### SUMMARY

Embodiments of the present disclosure provide benefits and/or solve one or more of the foregoing or other problems in the art with systems, non-transitory computer-readable media, and methods that utilize separability and/or disjointedness interaction models to analyze digital representations of cellular perturbations (e.g., machine learning embeddings) for determining measures of biological activity between perturbations. The disclosed system can also utilize these measures of biological activity for active learning and selection of samples for exploring underlying feature spaces. In particular, in one or more implementations the disclosed systems compare sets of individual perturbation representations to identify new pairwise interactions. In some embodiments, the disclosed systems can compare predicted pairwise representations with observed pairwise representations to determine a latent variable separability measure between a first perturbation and a second perturbation. For example, in some embodiments, the disclosed systems can determine the latent variable separability measure by comparing observed pairwise perturbation representations with predicted pairwise representations. To illustrate, the disclosed systems can generate and/or compare log-density ratio metrics (e.g., KL-divergence) and/or score function metrics (e.g., Fisher Divergence metrics or Kernelized Stein Discrepancy metrics). In this manner, the disclosed systems can determine a latent variable separability measure that indicates the amount of new information gained from observed pairwise representations relative to individual pairwise representations.

Moreover, in one or more embodiments, the disclosed systems can compare predicted pairwise representations with observed pairwise representations to determine a domain disjointedness measure between a first perturbation and a second perturbation. For example, in one or more embodiments, the disclosed systems can determine a mean discrepancy metric (e.g., maximum mean discrepancy) between an observed pairwise representation distribution and a synthetic pairwise perturbation distribution. In this manner, the disclosed systems can determine a domain disjointedness measure that indicates compositional generalization (e.g., the extent to which embeddings of two perturbations compose additively to predict pairwise perturbations).

Additionally, the disclosed systems can utilize active learning approaches to identify new pairwise perturbations to explore in identifying additional, previously undetected biological interactions. For example, in some embodiments, the disclosed systems efficiently search a space of perturbation pairs to identify new perturbation pairs for further exploration. Specifically, in one or more implementations, the disclosed systems utilize one or more active learning approaches to select an additional perturbation pair for further pairwise experimentation. To illustrate, in one or more implementations, the disclosed systems generate a perturbation interaction matrix and utilize an active-matrix completion algorithm to identify perturbation pairs that are most likely to have meaningful interactions (e.g., a biological activity beyond that expected from the individual perturbations themselves). Additionally, from the identified perturbation pair, the disclosed systems initiate performance of a downstream experiment for a cell to be exposed to the perturbation pair. In this manner, the disclosed systems can efficiently and accurately utilize perturbation representations, such as machine learning embeddings, to explore and identify previously unknown biological interactions between perturbations.

Additional features and advantages of one or more embodiments of the present disclosure are outlined in the description which follows, and in part will be obvious from the description, or may be learned by the practice of such example embodiments.

### BRIEF DESCRIPTION OF THE DRAWINGS

The detailed description provides one or more embodiments with additional specificity and detail through the use of the accompanying drawings, as briefly described below.

FIG. 1 illustrates an overview diagram of a multi-perturbation interaction system determining measures of biological activity of perturbations in accordance with one or more embodiments.

FIGS. 2A-2C illustrates an example diagram of the multi-perturbation interaction system generating sets of individual perturbation representations in accordance with one or more embodiments.

FIG. 3 illustrates an example diagram of the multi-perturbation interaction system generating distribution density ratios and utilizing the distribution density ratios to generate a synthetic pairwise distribution representation in accordance with one or more embodiments.

FIG. 4 illustrates an example diagram of the multi-perturbation interaction system utilizing feature vectors to generate Kullback-Leibler divergence metrics (e.g., latent variable separability measures) in accordance with one or more embodiments.

FIG. 5 illustrates an example diagram of the multi-perturbation interaction system utilizing score functions to generate a latent variable separability measure in accordance with one or more embodiments.

FIGS. 6A-6B illustrate an example diagram of the multi-perturbation interaction system mapping score functions to reproducing kernel spaces to generate Kernelized Stein Discrepancy metrics (e.g., latent variable separability measures) in accordance with one or more embodiments.

FIG. 7 illustrates an example diagram of the multi-perturbation interaction system determining a mean distance of feature embeddings to determine a domain disjointedness measure in accordance with one or more embodiments.

FIG. 8 illustrates an example diagram of the multi-perturbation interaction system mapping a synthetic pairwise perturbation distribution to a reproducing kernel space to determine a mean discrepancy metric in accordance with one or more embodiments.

FIG. 9 illustrates an example diagram of the multi-perturbation interaction system performing an active learning approach by utilizing a pairwise prediction model to select an entry from a matrix for further exploration in accordance with one or more embodiments

FIG. 10 illustrates an example diagram of a perturbation interaction matrix that includes multiple measures of biological activity and a plurality of predicted pairwise perturbation interaction scores in accordance with one or more embodiments.

FIG. 11 illustrates an example diagram of the multi-perturbation interaction system initiating downstream experimentation for a perturbation pair that corresponds with a selected entry from the perturbation interaction matrix in accordance with one or more embodiments.

FIGS. 12A-12E illustrates example experimental results of experimental implementations of the multi-perturbation interaction system in accordance with one or more embodiments.

FIG. 13 illustrates an example environment of the multi-perturbation interaction system in accordance with one or more embodiments.

FIG. 14 illustrates an example series of acts to generate a latent variable separability measure in accordance with one or more embodiments.

FIG. 15 illustrates an example series of acts to generate a domain disjointedness measure in accordance with one or more embodiments.

FIG. 16 illustrates a block diagram of a computing device for implementing one or more embodiments.

### DETAILED DESCRIPTION

Embodiments of the present disclosure provide benefits and/or solve one or more of the foregoing or other problems in the art with systems, non-transitory computer-readable media, and methods of a framework that determines separability and/or disjointedness measures from pairwise interaction representations of cellular perturbations as measures of biological activity between perturbations (e.g., for intelligent exploration of a complex perturbation interaction feature space). For example, the multi-perturbation interaction system uses sets of individual perturbation representations and observed pairwise representations to determine measures of biological activity of pairwise perturbations, such as a latent variable separability measure or a domain disjointedness measure. To illustrate, the multi-perturbation interaction system generates representations, such as digital images, embeddings, or transcriptomic profiles, of single perturbations. In some embodiments, the multi-perturbation interaction system then combines the representations of the individual perturbations to generate predicted pairwise representations, such as synthetic pairwise distribution representations (or synthetic pairwise perturbation distributions), for a pairwise perturbation. Further, in some embodiments, the multi-perturbation interaction system compares the predicted pairwise representations with observed pairwise representations to determine a measure of biological activity (e.g., an interaction score).

Pairwise interactions between perturbations to a system can provide evidence for the causal dependencies of the underlying mechanisms. When observations are low dimensional measurements, however, it is difficult to detect interactions between perturbations affecting latent variables. For example, in biology, performing a first CRISPR gene knockout on a first cell can cause a first cellular change (e.g., a change to a first cellular organelle) and performing a second CRISPR gene knockout on a second cell can cause a second cellular change (e.g., a change to a second cellular organelle). However, performing both the first CRISPR gene knockout and the second CRISPR gene knockout on the same cell can result in wildly different outcomes (e.g., death of the cell). Discovering such pairwise interactions can provide valuable insights into the underlying mechanisms of cellular interaction. The multi-perturbation interaction system can utilize interaction models to determine various unique biological activity metrics (e.g., latent variable separability measures and/or domain disjointedness measures) that indicate these unexpected pairwise interactions.

Moreover, the multi-perturbation interaction system can integrate these biological activity metrics into an active learning pipeline to efficiently discover pairwise interactions between perturbations. In some embodiments, the multi-perturbation interaction system generates measure of biological activity that indicate a comparison, or difference, between predicted pairwise representations and observed pairwise perturbation representations. Specifically, the multi-perturbation interaction system can use a latent variable separability measure to identify pairwise perturbations (e.g., such as gene knockouts) that have an additional biological interaction (e.g., a biological activity beyond that expected from the individual knockouts themselves). Moreover, the multi-perturbation interaction system can use the domain disjointedness measure to determine when a double perturbation to a cell or a group of cells can be predicted from a single perturbation. Further, the multi-perturbation interaction system can also utilize active learning to intelligently explore the perturbation feature space and select additional perturbation pairs for further exploration from these measures of biological activity.

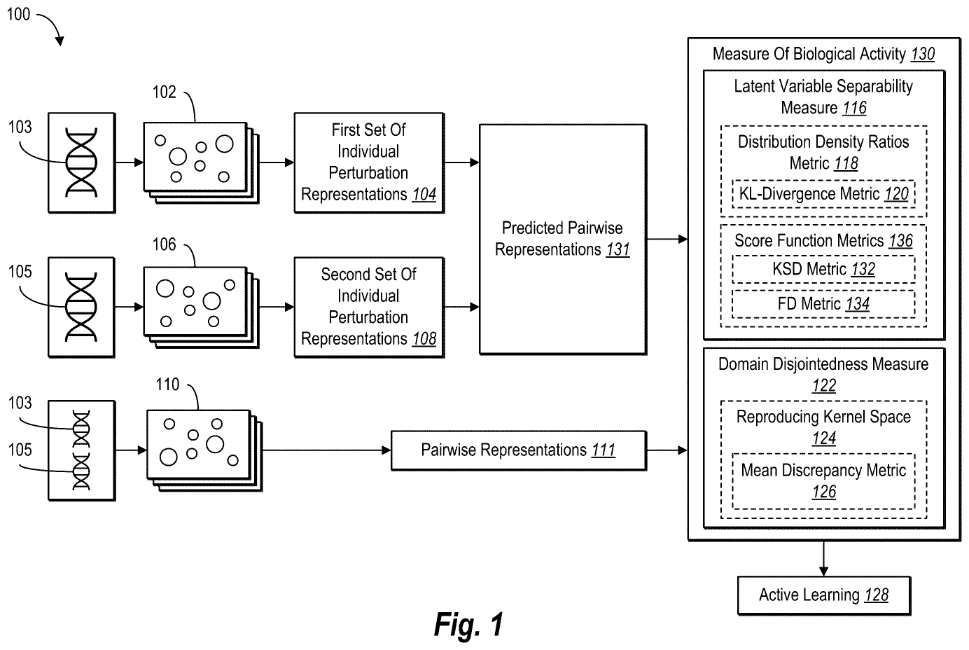

As shown, FIG. 1 illustrates a multi-perturbation interaction system 100 determining a measure of biological activity 130 for pairwise perturbations performed on sets of cells. The multi-perturbation interaction system 100 can determine the measure of biological activity 130 utilizing one or more testable relationships, and can generate one or more representations of cells exposed to perturbations to enable the multi-perturbation interaction system 100.

Specifically, FIG. 1 illustrates a first set of cells 102 exposed to a first perturbation 103 (e.g., a first gene knockout), a second set of cells 106 exposed to a second perturbation 105 (e.g., a second gene knockout), and a third set of cells 110 exposed to a combination of the first perturbation 103 and the second perturbation 105. The multi-perturbation interaction system 100 can combine the first individual set of perturbation representations 104 and the second individual set of perturbation representations 108 in different ways to form different types of predicted pairwise representations 131. For example, in some embodiments, the multi-perturbation interaction system 100 can combine the first individual set of perturbation representations 104 and the second individual set of perturbation representations 108 multiplicatively to generate a synthetic pairwise distribution representation. In some embodiments, the multi-perturbation interaction system 100 can combine the first individual set of perturbation representations 104 and the second individual set of perturbation representations 108 additively to determine a synthetic pairwise perturbation distribution.

In addition to generating the predicted pairwise representations 131 (e.g., by combining the first individual set of perturbation representations 104 and the second individual set of perturbation representations 108) the multi-perturbation interaction system 100 can generate pairwise representations 111 by exposing the third set of cells 110 to both the first perturbation 103 and the second perturbation 105. Responsive to exposing the third set of cells to the first perturbation 103 and to the second perturbation 105, the multi-perturbation interaction system 100 can generate different types of pairwise representations 111. For example, the multi-perturbation interaction system 100 can generate a set of observed pairwise perturbation representations of the third set of cells 110. Additionally or alternatively, the multi-perturbation interaction system 100 can generate observed pairwise representation distributions.

Responsive to generating the predicted pairwise representations 131 and the pairwise representations 111, the multi-perturbation interaction system 100 can compare the predicted pairwise representations 131 and the pairwise representations 111 to determine a measure of biological activity 130 (e.g., of the pairwise perturbations). For example, the multi-perturbation interaction system 100 can generate the measure of biological activity 130 by generating a latent variable separability measure 116 and/or a domain disjointedness measure 122.

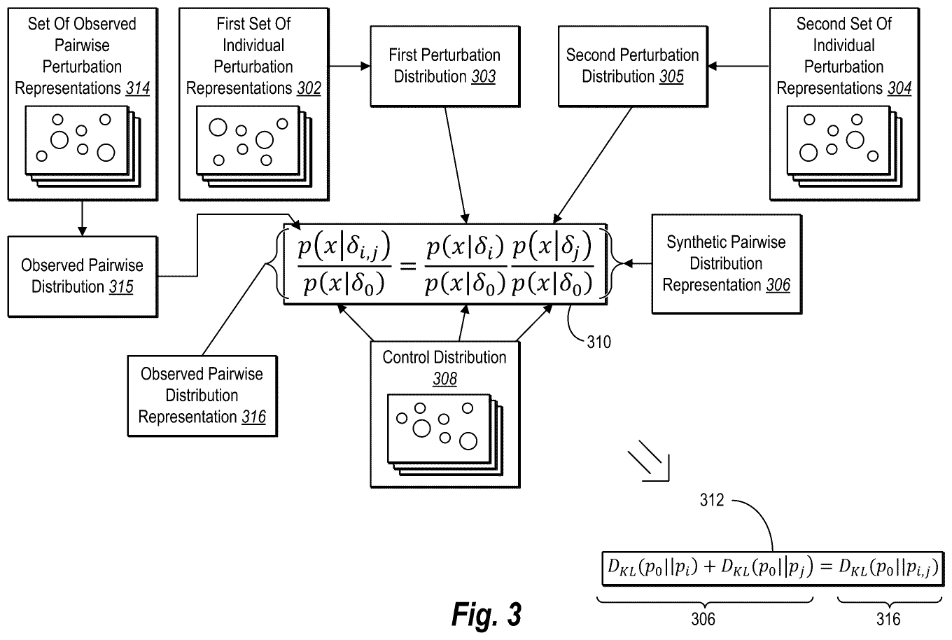

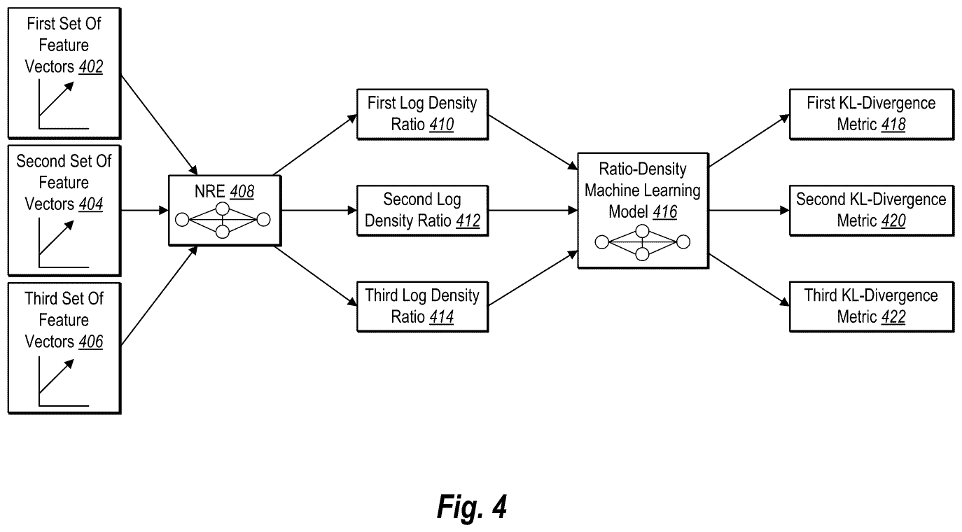

Indeed, in some embodiments, the multi-perturbation interaction system 100 can generate the latent variable separability measure 116 (e.g., a measure of separability of the first perturbation and the second perturbation) by comparing the synthetic pairwise distribution representation and the set of observed pairwise perturbation representations. Specifically, when generating the latent variable separability measure 116, the multi-perturbation interaction system 100 can utilize a distribution density ratio metric 118. For instance, the multi-perturbation interaction system 100 can generate a synthetic pairwise distribution representation comprising a combination of distribution density ratios (relative to a control distribution) for individual perturbation distributions. The multi-perturbation interaction system 100 can also generate an observed pairwise distribution representation comprising a distribution density ratio (relative to a control distribution) of the observed pairwise perturbation representations. By comparing these distribution density ratios, the multi-perturbation interaction system 100 can determine the latent variable separability measure 116. For example, in some implementations, the multi-perturbation interaction system 100 determines Kullback-Leibler divergence metrics 120 (a “KL-divergence metric 120”) and utilizes these metrics to generate the latent variable separability measure 116. Additional detail regarding latent variable separability measures and density distribution ratio metrics are provided below (e.g., in relation to FIGS. 3-4).

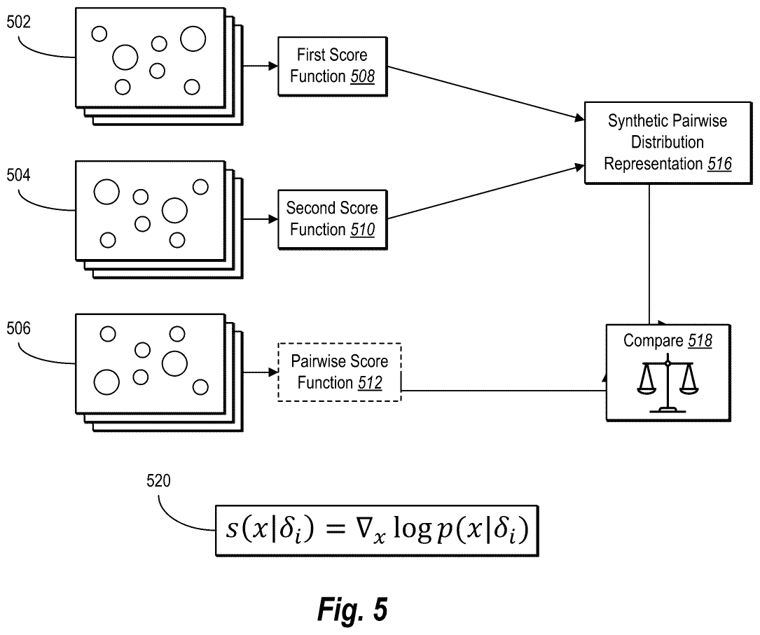

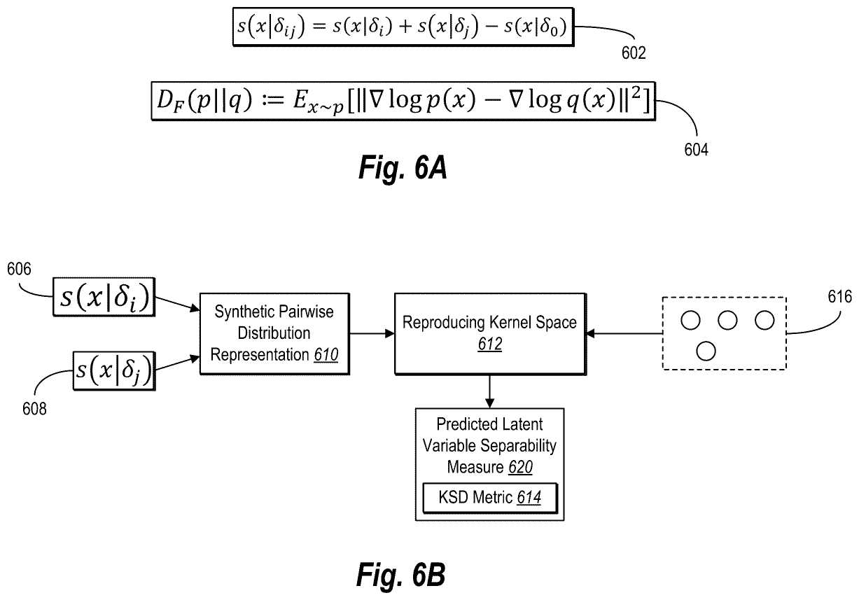

In addition, as shown, the multi-perturbation interaction system 100 can also generate one or more score function metrics 136. In particular, the multi-perturbation interaction system 100 can utilize a score function that indicates a gradient of a probability density function (e.g., a gradient of the log of the probability density function of a data distribution). The multi-perturbation interaction system 100 can compare score functions for different distributions to determine the latent variable separability measure 116. For example, in some embodiments, the multi-perturbation interaction system 100 generates a Kernelized Stein Discrepancy (KSD) metric 132 from the score functions. Similarly, in some implementations, the multi-perturbation interaction system 100 utilizes a Fisher Divergence (FD) metric 134 from the score functions. Additional detail regarding latent variable separability measures and score function metrics is provided below (e.g., in relation to FIG. 5-6B).

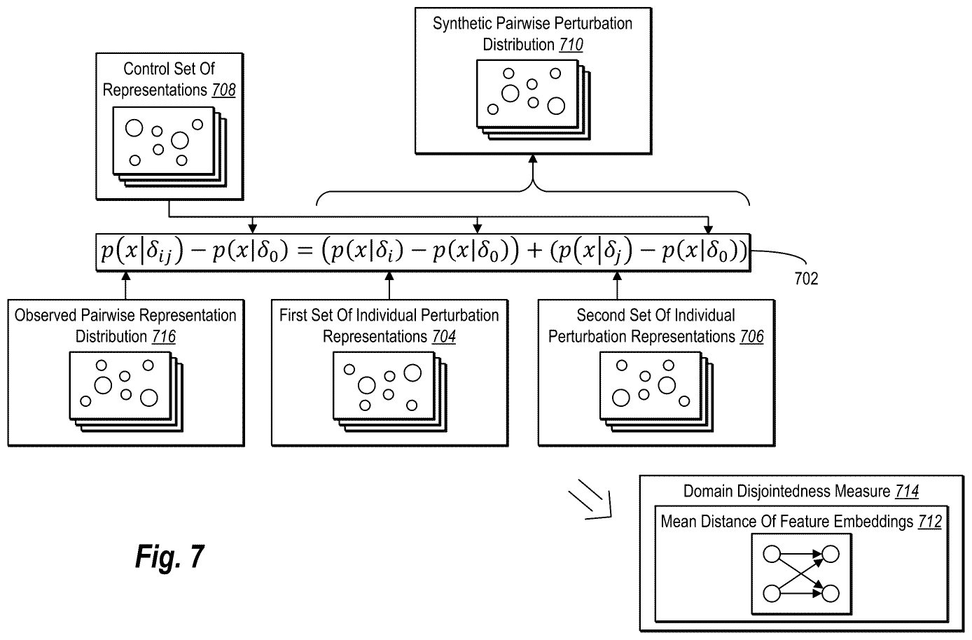

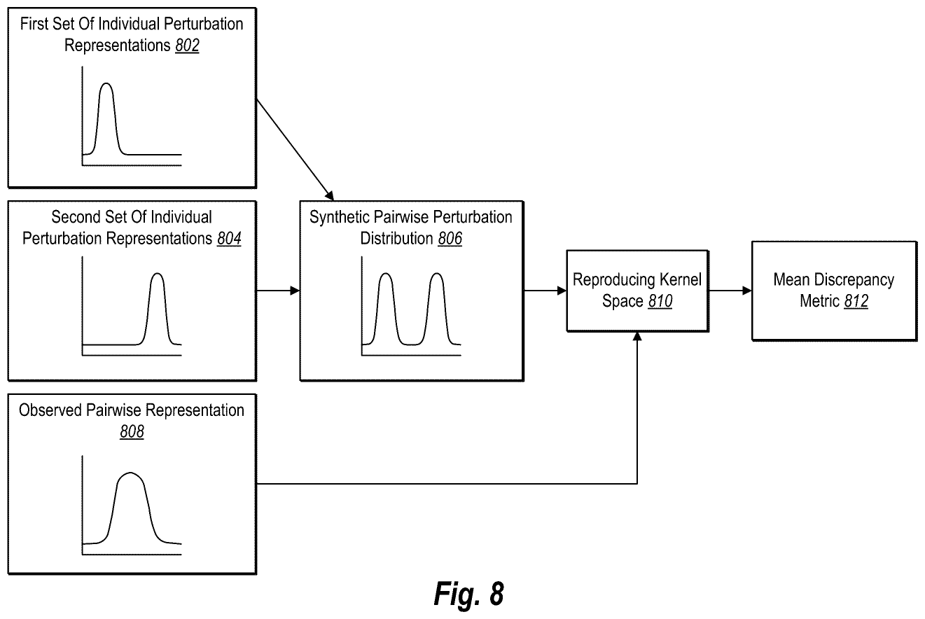

Further, in some embodiments, the multi-perturbation interaction system 100 can determine the domain disjointedness measure 122 (e.g., a measure of disjointedness of the first perturbation and the second perturbation) by comparing the synthetic pairwise perturbation distribution and the observed pairwise representation distribution. For instance, the multi-perturbation interaction system 100 can map the synthetic pairwise perturbation distribution and the observed pairwise representation distribution to a reproducing kernel space 124 to determine a mean discrepancy metric 126 between the synthetic pairwise perturbation distribution and the observed pairwise representation distribution. Additional detail regarding the domain disjointedness measure is provided below (e.g., in relation to FIG. 7-8).

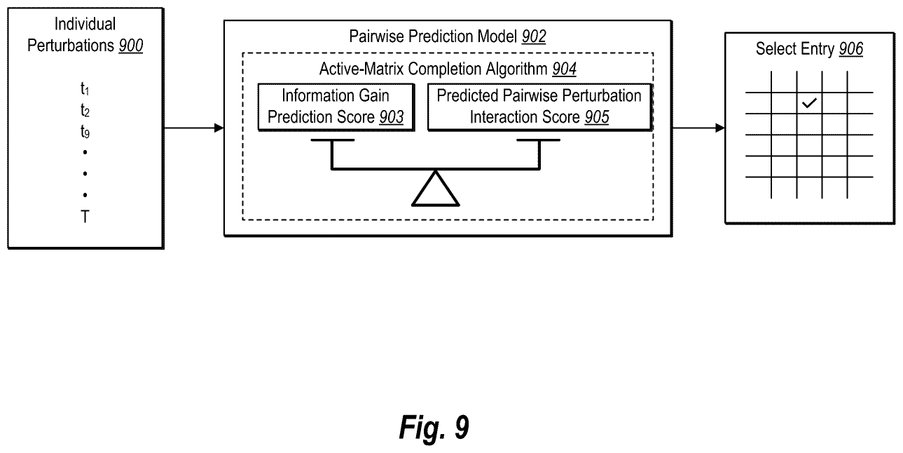

Further, as shown, responsive to determining the measure of biological activity 130, the multi-perturbation interaction system 100 can utilize active learning 128 to identify additional pairwise perturbations. For example, the multi-perturbation interaction system can avoid running experimentation for all perturbation pairs (e.g., all pairs of gene knockouts) to recover pairwise biological relationships. For instance, the multi-perturbation interaction system can generate a perturbation interaction matrix of different perturbations and corresponding measures of biological activity such as latent variable separability measures and/or domain disjointedness measures (e.g., for already known pairwise perturbations). Further, the multi-perturbation interaction system can efficiently explore the interaction space using active learning approaches. For example, by treating the measure of biological activity as a reward, the multi-perturbation interaction system can reduce the problem of finding additional perturbation pairs to an active-matrix completion problem. In some implementations, the multi-perturbation interaction system balances exploration and exploitation to select different entries (e.g., perturbation pairs) for initiating downstream experimentation. Additional detail regarding the multi-perturbation interaction system 100 utilizing an active learning pipeline is provided below (e.g., in relation to FIGS. 9-11).

As mentioned briefly above, conventional systems suffer from a number of technical deficiencies with regard to implementing computing devices. For example, conventional systems that perform double perturbation experiments are extremely inefficient with regard to time, computational resources, and memory expenditures for implementing computing devices in the genetic space. For instance, running double knockout gene experiments for perturbation pairs across the genome (and/or perturbation assays across the compound space) could include utilizing automated laboratories and implementing computing devices to implement, process, store, and manage hundreds of millions of experiments and corresponding digital results. Accordingly, conventional systems often require significant time, robotics equipment, memory, and/or processing power to analyze perturbation combinations. Further, conventional systems that attempt to detect pairwise interactions from unstructured data face expensive computational costs that are quadratic in the number of required perturbations.

Moreover, conventional systems often suffer from computational inaccuracies. Indeed, even after running computationally expensive assays, conventional systems often fail to accurately identify unique biological interactions that occur as a result of multiple perturbations. Indeed, unique interactions can be extremely nuanced, detailed, and difficult to detect. Thus, utilizing conventional systems to analyze perturbations fails to provide an accurate reflection of unique interactions between multiple perturbations.

Moreover, conventional systems suffer from operational inflexibility due to conventional systems being limited in identifying only certain biological interactions. As previously mentioned, conventional systems are often confined to data involving single perturbations and as such, fail to identify more complex relationships or interactions.

In one or more embodiments, the multi-perturbation interaction system 100 overcomes the deficiencies of conventional systems. For example, in some embodiments, the multi-perturbation interaction system 100 overcomes the inefficiencies of conventional systems by utilizing novel interaction models to extract unique measures of biological interactions, thus reducing the time, computational resources, and memory expenditures required to identify pairwise interactions. For example, the multi-perturbation interaction system 100 can efficiently generate latent variable separability measures and/or domain disjointedness measures that reflect separability and disjointedness of pairwise perturbation representations. These metrics provide a unique signal regarding the underlying latent variables and domain that impact multi-perturbation biological activity. This allows implementing systems to efficiently identify unique perturbation interactions and further select future interactions to explore.

Additionally, the multi-perturbation interaction system 100 utilizes active learning approaches to efficiently identify additional perturbation pairs that are likely to exhibit significant biological interactions. To illustrate, the multi-perturbation interaction system 100 generates a measure of biological activity for a pairwise perturbation and utilizes active-matrix completion (or another active learning approach) to further identify meaningful biological relationships. For instance, the multi-perturbation interaction system 100 identifies additional meaningful biological interactions utilizing an active-matrix completion algorithm, which enables the multi-perturbation interaction system 100 to avoid brute-force searches and instead intelligently select perturbation pairs based on a tradeoff between exploration (e.g., information gain) and exploitation (e.g., reward). Indeed, the multi-perturbation interaction system 100 can significantly reduce the time, computational resources, and memory requirements of running upwards of hundreds of millions of experiments. Rather, the multi-perturbation interaction system 100 can selectively identify perturbation pairs with high potential for biological activity beyond that expected from the individual perturbations themselves.

Additionally, the multi-perturbation interaction system 100 further overcomes inaccuracies of conventional systems by quantifying measures of biological activity for representations of combinations of perturbations performed on cells. Indeed, by computing latent variable separability measures and/or domain disjointedness measures for representations of combinations of perturbations performed on cells, the multi-perturbation interaction system 100 objectively and accurately identifies meaningful biological activities resulting from combinations of perturbations performed on cells. Moreover, the multi-perturbation interaction system 100 can accurately infer additional perturbation combinations that are likely to result in unique biological interactions.

Related to the efficiency and accuracy improvements, the multi-perturbation interaction system 100 further improves upon operational flexibility. Specifically, the multi-perturbation interaction system 100 expands the identification of meaningful biological interactions to pairwise perturbations (rather than just individual perturbations) and further searches a state space more efficiently by selecting perturbation pairs based on the criteria that balances reward and information gain.

As suggested by the foregoing, this application utilizes a variety of terms to describe the improvements and functions of the multi-perturbation interaction system 100. For example, as used herein, the term “perturbation” (e.g., cell perturbation) refers to an alteration or disruption to a cell or the cell's environment (to elicit potential phenotypic changes to the cell). In particular, the term perturbation can include a gene perturbation (i.e., a gene-knockout perturbation) or a compound perturbation (e.g., a molecule perturbation or a soluble factor perturbation). These perturbations are accomplished by performing a perturbation experiment. A perturbation experiment refers to a process for a perturbation to a cell. A perturbation experiment also includes a process for developing/growing the perturbed cell into a resulting phenotype.

In addition, the term “individual perturbation” refers to a particular perturbation (applied to one or more biological cells). Specifically, the multi-perturbation interaction system 100 performs a perturbation experiment that involves an individual perturbation to the cell. For example, the multi-perturbation interaction system 100 performs a single gene knockout on the cell.

Furthermore, the term “individual perturbation representation” (or perturbation representations or individual perturbation representations) refers to a cell representation resulting from an individual perturbation to a cell. The individual perturbation representation can be an image (i.e., a digital image the multi-perturbation interaction system 100 captures after performing the individual perturbation) of the individual perturbation. Additionally or alternatively, the individual perturbation representation can be a numerical representation, such as a vector representation or an embedding of the individual perturbation to the cell. For example, the individual perturbation representation can include a vector representation of a perturbation image generated by a machine learning model (e.g., a convolutional neural network, masked autoencoder, or other machine learning embedding model). Accordingly, the individual perturbation representation can be a feature vector generated by application of various convolutional neural network layers (at different resolutions/dimensionality). Further, in some embodiments, the individual perturbation representation can be a transcriptomic profile of the individual perturbation (e.g., a set of RNA transcripts present in the cell after the multi-perturbation interaction system 100 applies the perturbation to the cell). For example, the multi-perturbation interaction system 100 can perform the individual perturbation, determine changes in gene expression (such as RNA or mRNA counts) caused by the individual perturbation, generate a perturbation interaction matrix from the changes in gene expression. The multi-perturbation interaction system 100 can provide the perturbation interaction matrix to a machine learning model to cause the machine learning model to generate an embedding of the changes in gene expression.

Moreover, as used herein, the term “set of individual perturbation representations” can refer to multiple representations (e.g., a digital image, an embedding, and/or a transcriptomic profile, among others) of individual perturbations. In some embodiments, the set of individual perturbation representations can include multiple individual perturbation representations of the same type (e.g., multiple digital images, multiple embeddings, or multiple transcriptomic profiles). In some embodiments, the set of individual perturbation representations can include a combination of different types of individual perturbation representations (e.g., a combination of digital images, embeddings, and/or transcriptomic profiles of the individual perturbation).

Additionally, in some embodiments, the multi-perturbation interaction system 100 can generate the synthetic pairwise distribution representation from one or more individual perturbation distribution density ratio metrics. As used herein, the term “individual perturbation distribution density ratio metric” refers to a density ratio between a distribution that corresponds to a set of individual perturbation representations and a control distribution that corresponds to a control set of representations of a control set of cells (e.g., a set of cells not exposed to any perturbations). Indeed, in some embodiments, the individual perturbation distribution density ratio metric can be a Kullback-Leibler divergence metric (or KL-divergence metric) generated from a log-density ratio. Indeed, the multi-perturbation interaction system 100 can generate the log-density ratio from a set of feature vectors of the set of individual perturbation representations.

Further, in some embodiments, the synthetic pairwise distribution representation can be a score function or a combination of score functions (e.g., a score function metric). As used herein, the term “score function” refers to a measurement of change of a probability density. Indeed, a score function can indicate a gradient of a probability density of observing a particular cellular state caused in response to a perturbation applied to a group of cells. Further, a score function can indicate not only a magnitude of changes caused to a group of cells by a perturbation but also a direction that the perturbation shifts a probability distribution of the group of cells' molecular state.

Moreover, as used herein, the term “set of observed pairwise perturbation representations” refers to a representation of a set of cells exposed to a first perturbation and a second perturbation. The representation can be a digital image, an embedding, or a transcriptomic profile. Further, in some embodiments, the multi-perturbation interaction system 100 can utilize the set of observed pairwise perturbation representations to generate a pairwise score function. As used herein, the term “pairwise score function” refers to a score function refers to a measurement of an impact of a combination of perturbations on a group of cells.

Further, as used herein, the term “latent variable separability measure” refers to a measure of separability of a first perturbation and a second perturbation. Indeed, the latent variable separability measure can indicate a compounding effect to a group of cells that are exposed to both a first perturbation and a second perturbation as opposed to a group of cells that is exposed to one of the first perturbation or the second perturbation. In particular, a latent variable separability measure includes a measure or indication of interaction between two perturbations (e.g., in addition to biological impacts of the individual perturbations). In other words, the latent variable separability measure can indicate biological activity beyond that expected from the individual perturbations themselves, such as synthetic lethality-style interactions (e.g., apoptosis) or morphological/phenomic cell changes that result from double perturbations. For example, in some embodiments, double gene knockouts result in specific changes to a cell that would not manifest (or would manifest to a different degree) from individual gene knockouts.

For example, the set of individual perturbation representations can be a distribution density ratio metric. Indeed, the density ratio metric can indicate a density ratio between a first set of individual perturbation representations and a control distribution that corresponds to a control set of representations of a control set of cells.

Additionally, in some embodiments, the latent variable separability measure can be a Kernelized Stein Discrepancy (KSD) metric. Indeed, the multi-perturbation interaction system 100 can generate the KSD metric by utilizing a kernel function (e.g., a linear kernel, a polynomial kernel, a Gaussian kernel, a LaPlacian kernel, a sigmoid kernel, a cosine similarity kernel, a Matern kernel, an exponential kernel, among others) to compare probability densities between the observed pairwise perturbation representations and the combined score function.

Further, as used herein, the term “domain disjointedness measure” refers to a level of composability (e.g., of a first perturbation and a second perturbation). For example, a domain disjointedness measure indicates a measure or degree to which individual perturbation representations compose additively. Indeed, the domain disjointedness measure indicates whether the first perturbation and the second perturbation act on disjoint sets of latent variables. Specifically, the domain disjointedness measure helps the multi-perturbation interaction system 100 identify when a double perturbation to a cell or a group of cells can be predicted from a single perturbation.

Although FIG. 1 discusses double perturbations or pairwise experimentation, in one or more embodiments, the multi-perturbation interaction system 100 deals with multi-perturbations for a single cell and multi-pair experimentation. Specifically, the multi-perturbation interaction system 100 receives a triplet representation (e.g., resulting from a triple perturbation to a single cell) and compares the triplet representation with a predicted triplet representation generated by combining individual perturbation representations to generate a measures of biological activity.

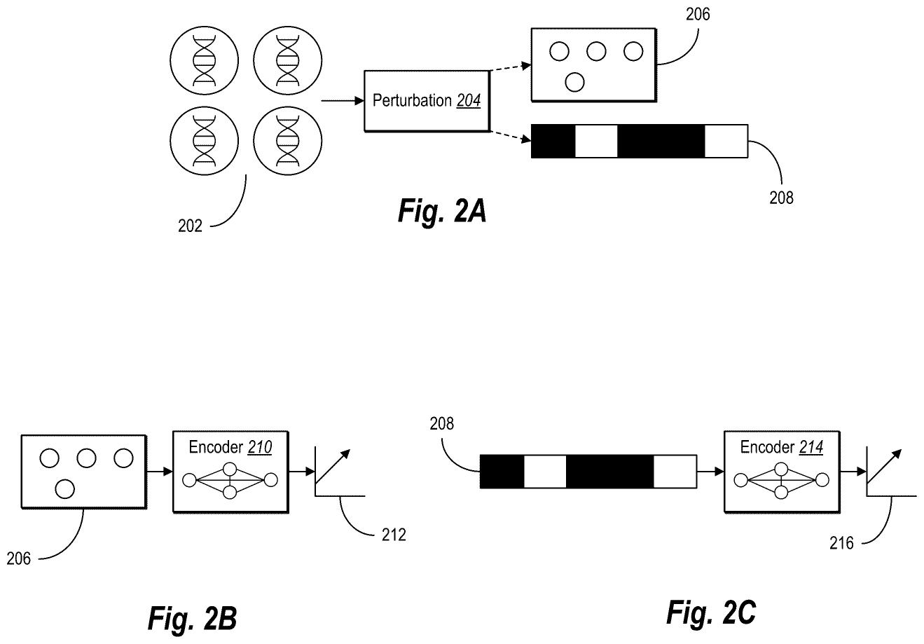

As previously mentioned, the multi-perturbation interaction system 100 generates sets of individual perturbation representations. FIGS. 2A-2C illustrates the multi-perturbation interaction system 100 exposing sets of cells to individual perturbations and generating sets of individual perturbation representations from the sets of cells.

As shown in FIG. 2A, the multi-perturbation interaction system 100 receives a set of cells 202. As used herein, the term “cell” refers to a structural, functional, and biological unit of living organisms. Specifically, a cell can vary in size shape, and function depending on the organism and the role of the cell. For example, a cell can include a plasma membrane to separate the internal cell environment from the external surroundings and the cell can further contain genetic material.

As shown in FIG. 2A, the multi-perturbation interaction system 100 exposes the set of cells 202 to a perturbation 204. Indeed, the multi-perturbation interaction system 100 can expose a plurality of sets of cells to a plurality of perturbations. For example the multi-perturbation interaction system 100 can expose a first set of cells to a first perturbation, a second set of cells to a second perturbation, and a third set of cells to a double perturbation (e.g., the multi-perturbation interaction system 100 exposes the third set of cells to the first perturbation and the second perturbation).

Responsive to exposing the set of cells 202 to the perturbation 204, the multi-perturbation interaction system 100 generates a set of individual perturbation representations. In some embodiments, the set of individual perturbation representations can be a digital image 206 of the set of cells 202 exposed to the perturbation (e.g., a digital image portraying a cell after applying the perturbation 204 to the cell). In some embodiments, the set of individual perturbation representations can be a transcriptomic profile 208 that quantifies changes in RNA counts (such as mRNA) of the set of cells 202 caused by exposure to the perturbation 204. In Some embodiments, the set of individual perturbation representations can be embeddings. FIGS. 2B-2C illustrate the multi-perturbation interaction system 100 generating embeddings from digital images and transcriptomic profiles, respectively.

As shown in FIG. 2B, the multi-perturbation interaction system 100 can utilize an encoder 210 to generate an embedding 212, such as a feature vector, of the digital image 206 (e.g., the digital image 206 of FIG. 2A). For example, the encoder 210 can be a convolutional neural network (CNN) comprising a plurality of layers. Indeed, the multi-perturbation interaction system 100 can utilize the encoder 210 to extract features from the digital image 206. Specifically, the multi-perturbation interaction system 100 can cause each of the plurality of layers of the encoder 210 to extract features from the digital image. The multi-perturbation interaction system 100 causes the encoder 210 to compress the features extracted from the digital image 206 and combines them (e.g., through dense layers and/or pooling operations) into a feature vector to form the embedding. The multi-perturbation interaction system 100 can perform this process iteratively (e.g., the multi-perturbation interaction system 100 can utilize the encoder 210 to generate a first embedding of a first digital image of a first set of cells exposed to a first perturbation, a second embedding of a second digital image of a second set of cells exposed to a second perturbation, a third embedding of a third set of cells exposed to the first perturbation and the second perturbation, and so on).

For example, in some embodiments, the multi-perturbation interaction system 100 can utilize an embedding model to generate phenomic embeddings of the sets of perturbations and then combine the phenomic embeddings (e.g., add, average, or otherwise combine the embeddings). For example, the multi-perturbation interaction system 100 can utilize an embedding model described in UTILIZING MACHINE LEARNING MODELS TO SYNTHESIZE PERTURBATION DATA TO GENERATE PERTURBATION HEATMAP GRAPHICAL USER INTERFACES, U.S. patent application Ser. No. 18/526,707, filed Dec. 1, 2023 (hereinafter application '707) or UTILIZING MASKED AUTOENCODER GENERATIVE MODELS TO EXTRACT MICROSCOPY REPRESENTATION AUTOENCODER EMBEDDINGS, U.S. patent application Ser. No. 18/545,399, filed Dec. 19, 2023, which is incorporated herein in its entirety (hereinafter application '399), which are incorporated by reference herein in their entirety.

Additionally or alternatively, as shown in FIG. 2C, the multi-perturbation interaction system 100 can utilize an encoder 214 to generate an embedding 216 of a transcriptomic profile 208 (e.g., the transcriptomic profile 208 of FIG. 2A). To illustrate, the multi-perturbation interaction system 100 can generate the transcriptomic profile 208 by utilizing computer hardware (e.g., a transcriptomics machine) to analyze perturbed cell. For example, in some embodiments, the multi-perturbation interaction system 100 can extract RNA from the perturbed cell and analyze the RNA. Indeed, the multi-perturbation interaction system 100 can process raw reads (e.g., raw sequencing data) into a usable format that accurately reflects gene expression levels. For example, the multi-perturbation interaction system 100 can process the raw reads using a series of steps that can include (but is not limited to): quality-checking the raw reads, aligning the raw reads to a reference genome or transcriptome using alignment tools (e.g., HISAT2 or STAR), to map each raw read to specific genes or exonic regions; quantifying the number of the raw reads corresponding to each gene (e.g., to quantify gene expression levels); and/or normalizing the data (e.g., to ensure comparability across samples). Moreover, the multi-perturbation interaction system 100 can generate the transcriptomic profile from the processed raw read of the perturbed cell.

Moreover, the multi-perturbation interaction system 100 can utilize the encoder 214 to learn features of the transcriptomic profile 208 and combine the features into the embedding 216. The multi-perturbation interaction system 100 can utilize a variety of machine learning models (e.g., transcriptomic machine learning models) to generate embeddings from transcriptomic profiles. For example, in some implementations, the multi-perturbation interaction system 100 utilizes scVI, Geneformer, scGPT, CellPLM, Universal Cell Embeddings or “UCE,” scBERT, and/or scVAEIT. The multi-perturbation interaction system 100 can also utilize Application '399 as an encoder for transcriptomic profiles.

As mentioned above, the multi-perturbation interaction system 100 can compare predicted pairwise representations with observed pairwise representations to determine biological interactions resulting from pairwise perturbations. For example, when generating sets of predicted pairwise representations and comparing the sets of predicted pairwise representations with observed pairwise representations, the multi-perturbation interaction system 100 has x observations in an observation space X of unstructured measurements such as pixels in an image, and a finite set of perturbations{Tᵢ∈Tᵢ: i∈[n]}. Specifically, perturbations can be binary perturbations, i.e., Tᵢ:={0, 1} for all i∈[n], and Ti=1 means perturbation i is applied. For all i, j∈[n], denote the perturbation indicator as follows:

$${δ}_{0} = \{ {T}_{[ n ]} = 0 \} {δ}_{i} = \{ {T}_{i} = 1 , {T}_{[ n ] \backslash \{ i \}} = 0 \} {δ}_{ij} = \{ {T}_{i} = {T}_{j} = 1 , {T}_{[ n ] ∖ \{ i , j \}} = 0 \}$$

The above assumes that for a pair of perturbations, T₁, Tⱼ, there is access to experimental data from four distributions p(x|δ₀), p(x|δᵢ), p(x|δⱼ), and p(x|δᵢⱼ). Further, it is appropriate to assume a set of latent variables {Z₁, . . . , Z_{L}⊆Z} in some latent space Z that capture relevant information about perturbations applied by the multi-perturbation interaction system 100. This assumption can be more precisely stated as X⊥⊥(T₁, . . . , Tₙ)|Z, or, more precisely:

$$p ( x \vert {T}_{1} , … , {T}_{n} ) = {\int }_{z} p ( x \vert z ) p ( z \vert {T}_{1} , … , {T}_{n} ) d z$$

Moreover, it is appropriate to assume all distributions have well-defined densities or probability mass functions with respect to some σ-finite base measure on their corresponding sample spaces. Further, distribution and density can be noted by the same symbol interchangeably.

As previously mentioned, the multi-perturbation interaction system 100 can generate distribution density ratios (e.g., distribution representations) and utilize the distribution density ratios to determine a latent variable separability measure for two perturbations. FIG. 3 shows the multi-perturbation interaction system 100 generating distribution density ratios from perturbation representations and utilizing the distribution density ratios to test for latent variable separability.

For example, one way the multi-perturbation interaction system 100 tests latent variable separability is by comparing density ratios between distributions (e.g., comparing distribution representations). For example, the multi-perturbation interaction system 100 can test separability by comparing density ratios between a synthetic pairwise distribution and an observed pairwise distribution. As shown in FIG. 3, this can be illustrated by equation 310 as follows:

$$\frac{p ( x \vert {δ}_{i , j} )}{p ( x \vert {δ}_{0} )} = \frac{p ( x \vert {δ}_{i} )}{p ( x \vert {δ}_{0} )} \frac{p ( x \vert {δ}_{j} )}{p ( x \vert {δ}_{0} )}$$

The multi-perturbation interaction system 100 can utilize the equation 310 to represent a relationship of separability between the first perturbation and the second perturbation. The multi-perturbation interaction system 100 can utilize one or more tests to measure or otherwise evaluate the relationship of separability between the first perturbation and the second perturbation. The multi-perturbation interaction system 100 can further utilize one or more methods to test the relationship of separability by utilizing one or more methods, such as determining a density ratio, determining a KL divergence metric, or determining a score function, among others. Indeed, by testing equation 310 for any particular perturbation pair, the multi-perturbation interaction system 100 can determine whether a first perturbation and a second perturbation are separable. For example, the difference between the left side of equation 310 and the right side of equation 310 would indicate a degree to which latent variables are separable (e.g., the difference indicates a latent variable separability measure). Accordingly, the multi-perturbation interaction system 100 can utilize this test to determine the separability of two perturbations.

To provide additional detail, FIG. 3 illustrates a first set of individual perturbation representations 302. For example, the first set of individual perturbation representations can include digital images, transcriptomic profiles, or embeddings as discussed above (e.g., in relation to FIGS. 2A-2C). The multi-perturbation interaction system 100 can generate a first perturbation distribution 303 from these representations (e.g., a probability distribution such as a probability density). Moreover, the multi-perturbation interaction system 100 can analyze this distribution to test for separability.

Indeed, as shown, the multi-perturbation interaction system 100 generates a first individual perturbation density ratio metric from the first set of individual perturbation representations 302 by determining a density ratio between the first perturbation distribution 303 that corresponds to the first set of individual perturbation representations 302 and a control distribution 308 that corresponds to a control set of representations of a control set of cells (e.g., a set of unperturbed cells).

The multi-perturbation interaction system 100 can generate the control distribution 308 by generating a representation of a set of unperturbed cells (e.g., a set of cells developed from the same batch without any perturbations applied to them). Moreover, in some embodiments, the multi-perturbation interaction system 100 can capture digital images of the set of unperturbed cells and utilize the digital images to generate embeddings of the set of unperturbed cells. In some embodiments, the multi-perturbation interaction system 100 can determine a transcriptomic profile of the set of unperturbed cells and utilize the transcriptomic profile to generate an embedding of the set of unperturbed cells.

Similarly, the multi-perturbation interaction system 100 generates a second perturbation distribution 305 (e.g., a probability distribution such as a probability density) from a second set of individual perturbation representations 304. Moreover, the multi-perturbation interaction system 100 generates a second individual perturbation density ratio metric by determining a density ratio between the second perturbation distribution 305 and the control distribution 308 that corresponds to a control set of representations of a control set of cells (e.g., a set of unperturbed cells in the perturbation assays for the second perturbation).

As shown, the multi-perturbation interaction system 100 combines the first individual perturbation density ratio metric (i.e., a first distribution representation) and the second individual perturbation density ratio (i.e., a second distribution representation) to form a combined perturbation density ratio. This combined perturbation density ratio reflects the synthetic pairwise distribution representation 306 for the first perturbation and the second perturbation.

Additionally, the multi-perturbation interaction system 100 can generate a set of observed pairwise perturbation representations 314. For example, the multi-perturbation interaction system 100 can perform a double perturbation (e.g., a first perturbation and a second perturbation) on a set of cells and generate a representation of the double perturbation (e.g., by generating an embedding of a digital image of the double perturbation or by generating an embedding of a transcriptomic profile of the double perturbation). In some embodiments, the set of observed pairwise perturbation representations 314 can be an embedding of the double perturbation.

As illustrated, the multi-perturbation interaction system 100 generates an observed pairwise distribution 315 (e.g., a probability distribution such as a probability density). Moreover, the multi-perturbation interaction system 100 generates a perturbation density ratio metric (e.g., an observed pairwise distribution representation 316) by determining a density ratio between the observed pairwise distribution 315 (e.g., a third density distribution) and the control distribution 308.

As shown in FIG. 3, the multi-perturbation interaction system 100 can compare the synthetic pairwise distribution representation 306 with the observed pairwise distribution representation 316 to determine a latent variable separability measure between the first perturbation and the second perturbation. Specifically, the multi-perturbation interaction system 100 can generate the synthetic pairwise distribution representation 306 as a statistical distribution representation of synthetically combining the effects of the first perturbation and the second perturbation when applied individually to separate sets of cells. Further, the multi-perturbation interaction system 100 can generate the observed pairwise distribution representation 316 as a statistical distribution representation of the observed effects of simultaneously applying the first perturbation and the second perturbation to a third set of cells. The multi-perturbation interaction system 100 can determine a difference between the synthetic pairwise distribution representation 306 and the observed pairwise distribution representation 316 to determine the latent variable separability measure.

It will be appreciated that the multi-perturbation interaction system 100 can test the equation 310 (and separability) in a variety of ways. For example, the multi-perturbation interaction system 100 can generate synthetic and observed pairwise distribution representations utilizing a variety of models or statistical approaches. Indeed, the term “synthetic pairwise distribution representation” can refer to a value, metric, or measure (e.g., a statistical representation) of a distribution (e.g., a distribution of samples from multiple perturbation). The synthetic pairwise distribution representation can reflect a distribution density ratio, a score function for a distribution, or other statistical metric for a pairwise distribution (e.g., a pairwise distribution resulting from combining samples of two individual perturbations). Thus, a synthetic pairwise distribution representation can include a statistical metric of a combined probability distribution resulting from a first perturbation performed on a first set of cells and a second perturbation performed on a second set of cells. Indeed, the multi-perturbation interaction system 100 can determine the synthetic pairwise distribution representation utilizing a variety of metrics, such as a distribution density ratio metric (e.g., a KL-divergence metric), or a score function metric (e.g., a kernelized Stein discrepancy metric or a Fisher Divergence metric). Similarly, an “observed pairwise distribution metric” can include a similar value, metric, or measure of a distribution of samples exposed to two perturbations. Additionally, in some embodiments, the synthetic pairwise distribution representation can be a mixture distribution that includes a combined probability distribution resulting from a first perturbation performed on a first set of cells and a second perturbation performed on a second set of cells, as well as one or more normalizing constants. For example, the multi-perturbation interaction system 100 can determine to add the one or more normalizing constants to the synthetic pairwise distribution representation to enable an integration of the synthetic pairwise distribution representation to yield a result of one.

For example, the bottom right corner of FIG. 3 illustrates utilizing KL-divergence metrics as a pairwise distribution representation. Specifically, FIG. 3 illustrates the multi-perturbation interaction system 100 generating the synthetic pairwise distribution representation 306 as a combination of KL-divergence metrics for the first perturbation and the second perturbation. Specifically, the multi-perturbation interaction system 100 generates a first KL-divergence metric (e.g., a first distribution representation) based on the first perturbation distribution 303 and the control distribution 308. Moreover, the multi-perturbation interaction system 100 generates a second KL-divergence metric (e.g., a second distribution representation) based on the second perturbation distribution 305 and the control distribution 308). The multi-perturbation interaction system 100 combines the first KL-divergence metric and the second KL-divergence metric to generate the synthetic pairwise distribution representation 306.

Similarly, FIG. 3 illustrates utilizing a KL-divergence metric to generate the observed pairwise distribution representation 316. Specifically, the multi-perturbation interaction system 100 generates the observed pairwise distribution representation 316 based on the observed pairwise distribution 315 and the control distribution 308. As shown in FIG. 3, the difference between these KL-divergence metrics (e.g., the difference between the left side and right side of equation 312) provides a latent variable separability measure equivalent/similar to the test of equation 310.

Specifically, the validity of equation 310 includes several mathematical assumptions. The first assumption is a variation of the change of variable formula. Namely, the first assumption states that there exists a diffeomorphism (e.g., a differentiable bijection with a differentiable inverse) g:Z→X such that X=g(Z). This assumption is valid for as long as the change of variable formula holds for the distributions of Z and X.

Based on the first assumption, when latent variables (e.g., such as a first set of independent perturbation representations and a second set of independent perturbation representations) are independent, the change of variable formula implies that the density ratio of a perturbed distribution to the original (e.g., control) distribution has a form that only involves the distribution of the intervened latent variable, according to the following equation:

$$\frac{p ( x \vert {δ}_{i} )}{p ( x \vert {δ}_{0} )} = \frac{{p}_{z} ( {g}^{- 1} ( x ) \vert {δ}_{i} ) \vert det ( J ( {g}^{- 1} ) ( x ) ) ) \vert}{{p}_{z} ( {g}^{- 1} ( x ) \vert {δ}_{0} ) \vert det ( J ( {g}^{- 1} ) ( x ) ) ) \vert} = \frac{{p}_{{z}_{l}}^{†} ( {g}^{- 1} ( x ) )}{{p}_{{z}_{l}} ( {g}^{- 1} ( x ) )}$$

### Where

$${p}_{{z}_{l}}^{†}$$

denotes the perturbed distribution of the latent variable Zᵢ targeted by intervention i. The above equation enables the determination that when two variables (e.g., a first set of individual perturbation representations and a second set of individual perturbation representations) are independent, the multi-perturbation interaction system 100 can predict the density ratio of the double perturbation (e.g., a first perturbation and a second perturbation applied to a third set of cells) as a product of the density ratios of the respective single perturbations. To increase the accuracy of this prediction, there is a further assumption that there is a causal factorization of the latent distribution, where each latent variable is conditionally independent of its non-descendants given its parents according to the following equation:

$${p}_{z} ( z \vert {T}_{1} , … , {T}_{N} ) = {\prod }_{l = 1}^{L} {p}_{{z}_{l}} ( {z}_{l} \vert P a ( {z}_{l} ) )$$

Where Pa(Y) (resp. Ch(Y)) denotes the parent (resp. children) of random variable Y. Further:

$$∀ l ∈ [ L ] , P a ( {Z}_{l} ) ⊆ {T}_{[ n ]} ⋃ {Z}_{[ L ]}$$

where the random variable Z represents the concatenation of all latent factors. Additionally, the random variable Z can be interpreted as a concatenation of latent factors {Z₁, . . . , Z_{L}}.

More information will now be provided regarding the equation

$${p}_{z} ( z \vert {T}_{1} , … , {T}_{N} ) = {\prod }_{l = 1}^{L} {p}_{{z}_{l}} ( {z}_{l} \vert P a ( {z}_{l} ) )$$

and how the change of variable formula can be applied to it to obtain the log density of p_{X} (x|T₁, . . . , Tₙ). Specifically,

$$log p ( x \vert {T}_{1} , … , {T}_{n} ) = {\sum }_{l = 1}^{L} log {p}_{{z}_{l}} ( {[ {g}^{- 1} ( x ) ]}_{l} \vert Pa ( {z}_{l} ) ) + log \vert det {∇}_{{g}^{- 1}} ( x ) \vert$$

In the foregoing, [⋅]ₗ can denote a projection operator that maps z∈Z to the subspace on which zₗ lies, i.e., ∀z∈Z, [z]ₗ=zₗ. Indeed, z can be interpreted as a concatenation of latent factors (zₗ, . . . , z_{L}). If Tᵢ, Tⱼ are causally independent, perturbation δᵢ,δⱼ can affect different terms in the foregoing equation. Without loss of generality, suppose δi and δⱼ intervene zₗᵢ and zₗⱼ respectively. Then,

$${\overline{ℓ}}_{X} ( x \vert {δ}_{i} ) = log p ( x \vert {δ}_{i} ) - log p ( x \vert {δ}_{0} ) = log {p}_{{z}_{l}} ( {[ {g}^{- 1} ( x ) ]}_{{l}_{i}} \vert {δ}_{i} ) - log {p}_{{z}_{{l}_{i}}} ( {[ {g}^{- 1} ( x ) ]}_{{l}_{i}} \vert {δ}_{0} ) .$$

Where the foregoing equality follows from a modularity assumption that an intervention only affects lᵢ. Specifically, it can be determined that

$${\overline{ℓ}}_{X} ( x \vert {δ}_{i} ) = log {p}_{{z}_{{l}_{j}}} ( {[ {g}^{- 1} ( x ) ]}_{{l}_{i}} \vert {δ}_{j} ) - log {p}_{{z}_{{l}_{j}}} ( {[ {g}^{- 1} ( x ) ]}_{{l}_{j}} \vert {δ}_{0} )$$

$${\overline{ℓ}}_{X} ( x \vert {δ}_{i j} ) = log {p}_{{z}_{{l}_{j}}} ( {[ {g}^{- 1} ( x ) ]}_{{l}_{i}} \vert {δ}_{i} ) - log {p}_{{z}_{{l}_{j}}} ( {[ {g}^{- 1} ( x ) ]}_{{l}_{i}} \vert {δ}_{0} ) + log {p}_{{z}_{{l}_{j}}} ( {[ {g}^{- 1} ( x ) ]}_{{l}_{j}} \vert {δ}_{j} ) - log {p}_{{z}_{{l}_{j}}} ( {[ {g}^{- 1} ( x ) ]}_{{l}_{j}} \vert {δ}_{0} ) .$$

Indeed, the multi-perturbation interaction system 100 utilizes the above equations to determine that the effects of two non-interacting perturbations are attributed to distinct causal pathways, rather than being confounded by a shared latent factor.

Further, according to the above discussion, the multi-perturbation interaction system 100 can determine that two perturbations δi,δj (e.g., a first set of individual perturbation representations and a second set of individual perturbation representations) are separable if Ch(Ti)∩Ch(Tj)=Ø, showing that when two perturbations act separably, the resulting density ratios (e.g., density ratio metrics) will have the predictable interactions as shown by the equation 310.

Further, the equation 310 can be manipulated to determine if the following holds true:

$$log p ( x \vert {δ}_{i j} ) + log p ( x \vert {δ}_{0} ) - log p ( x \vert {δ}_{i} ) - log p ( x \vert {δ}_{j} ) = 0$$

Additionally, the expectations of the above equation are applied to p(x|δ₀), which enables the multi-perturbation interaction system 100 to generate the first individual perturbation distribution density ratio metric by generating a first KL-divergence metric for the synthetic pairwise distribution representation and a second KL-divergence metric for the set of observed pairwise perturbation representations and compare the combined KL-divergence metric with the KL-divergence metric to determine a latent variable separability measure. Equation 312, which is reproduced below, illustrates comparing the first KL-divergence metric with the second KL-divergence metric to determine the latent variable separability measure:

$${D}_{K L} ( {p}_{0} ‖ {p}_{i} ) + {D}_{K L} ( {p}_{0} ‖ {p}_{j} ) = {D}_{K L} ( {p}_{0} ‖ {p}_{i , j} )$$

In the equation 312, pᵢ=p(x|δᵢ), p₀=p(x|δ₀), and D_{KL}(⋅∥⋅) denotes the KL-divergence metric. The multi-perturbation interaction system 100 utilizes the KL-divergence metric to measure the difference between two distributions. Specifically, the multi-perturbation interaction system 100 utilizes the KL-divergence metric to quantify the distribution shift resulting from perturbations. Indeed, the multi-perturbation interaction system 100 utilizes the KL-divergence metric to quantify the violation of the separability of the first perturbation and the second perturbation (e.g., an inequality in the equation 312 indicates that the first perturbation and the second perturbation are separable).

As previously mentioned, in some embodiments the multi-perturbation interaction system 100 can generate a distribution density ratio by generating a KL-divergence metric for a first set of individual perturbation representations. FIG. 4 illustrates the multi-perturbation interaction system 100 generating feature vectors to utilize as sets of individual perturbation representations, generating log-density ratios for the feature vectors, and generating KL-divergence metrics from the log-density ratios.

As discussed previously with regard to FIG. 2, the multi-perturbation interaction system 100 can utilize an encoder to generate sets of feature vectors to utilize as sets of individual perturbation representations. As shown in FIG. 4, the multi-perturbation interaction system 100 can generate a first set of feature vectors 402, a second set of feature vectors 404, and a third set of feature vectors 406.

Responsive to generating the sets of feature vectors, the multi-perturbation interaction system 100 can provide the sets of feature vectors to a machine learning model, such as a neural ratio estimator 408 (NRE 408). The NRE 408 can be a neural network that the multi-perturbation interaction system 100 trained (e.g., through training data) to compare samples from two distributions to determine a level of similarity of the two distributions. For example, the multi-perturbation interaction system 100 can train the NRE 408 as a contrastive learning model. Specifically, the multi-perturbation interaction system 100 can train a binary classifier to distinguish joint data distribution p(x, c) from the product of the marginals p(x)p(c), where c denotes the perturbation class.

Specifically, the multi-perturbation interaction system 100 can train the NRE 408 according to the following objective:

$$θ , w ∈ arg \begin{matrix}min \\ θ , w\end{matrix} - \frac{1}{2 B} [ {\sum }_{b = 1}^{B} log ( 1 - Sigmoid ( {f}_{θ , w} ( {x}^{( b )} , {c}^{( b )} ) ) + {\sum }_{{b}^{′} = 1}^{B} log ( Sigmoid ( {f}_{θ , w} ( {x}^{( {b}^{′} )} , {c}^{( b ′ )} ) ) ]$$

where x(b),c(b)~p(x)p(c) and x (b′),c(b′)~p(x, c), and fθ,W (x, c)=Encoderθ(x)T Wc.

As shown in FIG. 4, the multi-perturbation interaction system 100 can cause the NRE 408 to generate a first log-density ratio 410 (e.g., from the first set of feature vectors 402), a second log-density ratio 412 (e.g., from the second set of feature vectors 404) and a third log-density ratio 414 (e.g., from the third set of feature vectors 406). In one or more embodiments, the multi-perturbation interaction system 100 can utilize a 3-layer multi-layer perceptron (MLP) with ReLu Activation (and hidden dimensions of 2048 and 256) as an encoder for an NRE density ratio estimator utilizing an ADAM optimizer with a step size of 0.0001 for 2500 epochs and a batch size of 16384.

Further, the multi-perturbation interaction system 100 can provide the log-density ratios to a ratio-density machine learning model 416 to cause the ratio-density machine learning model 416 to generate KL-divergence metrics. Specifically, the multi-perturbation interaction system 100 can cause the ratio-density machine learning model 416 to determine a first KL-divergence metric 418, a second KL-divergence metric 420, and a third KL-divergence metric 422. The multi-perturbation interaction system 100 can generate the KL-divergence metrics in a plurality of ways.

For example, the multi-perturbation interaction system 100 can generate the KL-divergence metrics utilizing Monte Carlo Estimates based on samples from p(X|δ₀), i.e.,

$${D}_{KL} ( P ( X \vert {δ}_{i} ) ‖ P ( X \vert {δ}_{0} ) ) \approx \frac{1}{N} {\sum }_{i = I}^{N} f ( {X}_{i} )$$

where f(⋅) is the estimated log

$$\frac{p ( \cdot \vert {δ}_{i} )}{p ( \cdot \vert {δ}_{0} )} .$$

Alternatively, the multi-perturbation interaction system 100 can estimate the KL-divergence metric based on its Donkster-Varadhan representation according to the following equation:

$${D}_{K L} ( p ‖ q ) = \begin{matrix}sup \\ f : Ω \to ℝ\end{matrix} {𝔼}_{p} [ f ] - log {𝔼}_{q} [ exp ( f ) ]$$

where the supremum is taken over all functions ƒ such that the two expectations are finite. Indeed, the optimal ƒ is achieved as the log-density ratio between p and q.

Indeed, in some embodiments, the multi-perturbation interaction system 100 can first learn the log-density ratio (log p/q) as ƒ, and then estimate the KL-divergence metric according to the following equation:

$${KL}_{MILE} ( f ) = {𝔼}_{p} [ f ] - log {𝔼}_{q} [ exp ( f ) ]$$

The above equation can provide a lower-bound for the KL-divergence metric. Additionally, the above equation can be referred to as a mutual information lower-bound estimator.

Further, in one or more embodiments, the multi-perturbation interaction system 100 can clip the learned log-density ratios ƒ between −τ and τ to generate the following equation:

$${KL}_{SMILE} ( f , τ ) = {𝔼}_{p} [ f ] - log {𝔼}_{q} [ clip ( exp ( f ) , exp ( - τ ) , exp ( τ ) ) ]$$

As previously mentioned, in some embodiments, the multi-perturbation interaction system 100 can determine score functions for the sets of individual perturbation representations and utilizing score functions to determine latent variable separability measures. FIG. 5 illustrates the multi-perturbation interaction system 100 determining score functions for sets of individual perturbation representations and utilizing score functions to determine a latent variable separability measure.

Indeed, as shown in FIG. 5, the multi-perturbation interaction system 100 can generate a first set of individual perturbation representations 502 (e.g., a first representation of a first perturbation applied to a first set of cells), a second set of individual perturbation representations 504 (e.g., a second representation of a second perturbation applied to a second set of cells), and set of observed pairwise perturbation representations 506 (e.g., a third representation of the first perturbation and the second perturbation applied to a third set of cells). As previously discussed with regards to FIGS. 2A-2C, the sets of individual perturbation representations can be digital images, embeddings (e.g., feature vectors), or transcriptomic profiles.

As illustrated in FIG. 5, the multi-perturbation interaction system 100 can generate score functions for the sets of individual perturbation representations. Specifically, the multi-perturbation interaction system 100 can utilize a score function to indicate a gradient of a first probability density of a set of individual perturbation representations.

Specifically, the multi-perturbation interaction system 100 can generate a first score function 508 that indicates a first gradient of a first probability density of the first set of individual perturbation representations. Additionally, the multi-perturbation interaction system 100 can generate a second score function 510 that indicates a second gradient of a second probability density of the second set of individual perturbation representations. Further, the multi-perturbation interaction system 100 can combine the first score function 508 and the second score function 510 to generate a synthetic pairwise distribution representation 516.

Further, as shown, the multi-perturbation interaction system 100 can compare the synthetic pairwise distribution representation 516 (e.g., the combined score function) with the set of observed pairwise perturbation representations 506 to determine a predicted latent separability measure. Indeed, in some embodiments, the multi-perturbation interaction system 100 can generate a pairwise score function 512 (e.g., an observed pairwise distribution representation) from the set of observed pairwise perturbation representations 506.

Moreover, the multi-perturbation interaction system 100 can generate the latent variable separability measure by performing an act 518. Specifically, at the act 518, the multi-perturbation interaction system 100 can compare the synthetic pairwise distribution representation 516 with the set of observed pairwise perturbation representations 506. In some implementations, the multi-perturbation interaction system 100 performs the act 518 by comparing the synthetic pairwise distribution representation with the pairwise score function 512.

In some implementations, the multi-perturbation interaction system 100 omits the step of generating the pairwise score function 512. Indeed, the multi-perturbation interaction system 100 can compare individual observed pairwise perturbation representations with the synthetic pairwise distribution representation (e.g., the combined score function) rather than generating the pairwise score function 512. This can assist in reducing computational bandwidth and improve efficiency.

As shown in FIG. 5, the multi-perturbation interaction system 100 can utilize an equation 520, reproduced below, to generate the score functions:

$$s ( x \vert {δ}_{i} ) = {∇}_{x} log p ( x \vert {δ}_{i} )$$

More information will now be provided regarding the generation and use of score functions. Indeed, the use of score functions to determine a measure of separability of latent variables (e.g., perturbations) assumes that observation X obeys the following generative process involving latent random variable Z and noise variable U:

$${X}^{t} = f ( {Z}^{t} , U )$$

$$where$$

$${Z}^{t} ∼ {p}_{Z} ( z \vert T = t ) , U ∼ {p}_{U} , T ⊥ ⊥ U , T ⊥ Z , U ⊥ ⊥ Z$$

Indeed, T denotes a perturbation variable and t indexes experimental perturbations.

$${δ}_{i} := \{ T = i \} ( ∀ i ∈ [ n ] )$$

and

$$( \begin{matrix}n \\ 2\end{matrix} )$$

pairwise perturbations, denoted as

$${δ}_{i j} := {δ}_{i} ∩ {δ}_{j} ( i \neq j )$$

Further,

$${δ}_{0} = \{ T = 0 \}$$

represents the unperturbed environment, and T [symbol] Z, T⊥⊥U yield that the perturbation only intervenes with the latent variable Z but not the noise U, and the structural equation ƒ does not get intervened as well. Hence, the above equation

$${X}^{t} = f ( {Z}^{t} , U )$$

ensures that at X⊥⊥(T₁, . . . , Tₙ)|Z. Further, the above equations assume that Z admits a causal factorization, such that

$${p}_{Z} ( z ) = {\prod }_{l = 1}^{L} {p}_{{Z}_{l}} ( {z}_{l} \vert P a ( {z}_{l} ) )$$

$$where$$

$$∀ l ∈ [ L ] , Pa ( {Z}_{l} ) ⊆ \{ {Z}_{1} , … , {Z}_{L} \}$$

denotes that parent nodes of zₗ, the set of latent variables that causally influence Zₗ. Each perturbation targets a subset of latent variables, inducing a soft intervention, which changes the corresponding conditional distributions. For example, suppose that the latent variable Zₗ is targeted by the perturbation δᵢ, then

$${p}_{{Z}_{l}} ( {z}_{i} \vert P a ( {z}_{i} ) )$$

gets changed into,

$${p}_{{Z}_{i}}^{†} ( {z}_{i} \vert P a ( {z}_{i} ) )$$

$$hence$$

$${p}_{Z} ( z \vert {δ}_{i} ) = {p}_{{Z}_{i}}^{†} ( {z}_{i} \vert P a ( {z}_{i} ) ) \cdot {\prod }_{l \neq i} {p}_{{Z}_{i}} ( {z}_{l} \vert P a ( {z}_{l} ) )$$

Further, there is an assumption that there are n single perturbations, denoted as The above model leads to a natural interpretation of interactions between two perturbations: if two perturbations are non-interacting, multi-perturbation interaction system 100 will determine that each perturbation tar gets distinct (e.g., separate) latent factors. Indeed, the above model can provide a definition of separability as follows: denote I(t) the index of latent variables that are targeted by the perturbation t. Perturbations δᵢ, δₛ are separable if I(i)∩(j)=Ø. This leads to the following testable implication:

$$log \frac{p ( x \vert {δ}_{i} )}{p ( x \vert {δ}_{0} )} + log \frac{p ( x \vert {δ}_{j} )}{p ( x \vert {δ}_{0} )} + log \frac{p ( x \vert {δ}_{ij} )}{p ( x \vert {δ}_{0} )}$$

Further, a similar relationship can be derived that is based on score functions (e.g., the gradients of log-densities) rather than log-densities, given an injectivity condition on the structural equation ƒ. Specifically, the injectivity condition does not explicitly assume a diffeomorphism between the latent variable Z and the observation X Assuming that equation ƒ is injective, if perturbations δᵢ and δⱼ are separable, then the following separability of score functions equation holds:

$$s ( x \vert {δ}_{i j} ) = s ( x \vert {δ}_{i} ) + s ( x \vert {δ}_{j} ) - s ( x \vert {δ}_{0} )$$

Similar to equation 310, the foregoing equation can provide a test for separability. The left side of the equation reflects an observed pairwise distribution representation. The right side of the equation reflects a synthetic pairwise distribution representation (generated based on combining individual distribution representations or score functions for each perturbation).

As mentioned above, the multi-perturbation interaction system 100 can utilize a variety of models or formulations to test for separability, including a Fisher Divergence metric and/or a Kernelized Stein Discrepancy metric. Indeed, FIG. 6A illustrates testing separability of score functions utilizing a Fisher Divergence metric and FIG. 6B illustrates an application of the separability of score functions by mapping the score functions to a reproducing kernel space to determine a Kernelized Stein Discrepancy metric (KSD metric).

As shown in FIG. 6A, the multi-perturbation interaction system 100 can utilize an equation 602, reproduced below, to determine separability of a first perturbation and a second perturbation:

$$s ( x \vert {δ}_{i j} ) = s ( x \vert {δ}_{i} ) + s ( x \vert {δ}_{j} ) - s ( x \vert {δ}_{0} )$$

$$where$$

$$s ( x \vert {δ}_{i} ) = {∇}_{x} log p ( x \vert {δ}_{i} )$$

The multi-perturbation interaction system 100 can implement this separability tests utilizing a variety of models or metrics. For example, in one or more implementations, the multi-perturbation interaction system 100 utilizes equation 604, reproduced below:

$${D}_{F} ( p ‖ q ) := {E}_{x ∼ p} [ {‖ ∇ log p ( x ) - ∇ log q ( x ) ‖}^{2} ]$$

Indeed, the equation 604 illustrates Fisher Divergence (FD), which measures the discrepancy between two distributions by comparing their score functions.

As illustrated in FIG. 6B, the multi-perturbation interaction system 100 can also utilize score functions to determine a latent variable separability metric 620. Indeed, as previously discussed, the multi-perturbation interaction system 100 can generate a first score function 606 that indicates a first gradient of a first probability density of a first set of individual perturbation representations. Additionally, the multi-perturbation interaction system 100 can generate a second score function 608 that indicates a second gradient of a second probability density of a second set of individual perturbation representations. The multi-perturbation interaction system 100 can combine the first score function and the second score function (and account for a control score function, e.g., s(x|δ₀)) to form a synthetic pairwise distribution representation 610.

As shown in FIG. 6B, the multi-perturbation interaction system 100 can determine the latent variable separability metric 620 by generating a Kernelized Stein Discrepancy metric 614 (KSD metric 614). Indeed, the KSD metric 614 is a nonparametric measure that assesses a goodness-of-fit between a target distribution p and a model distribution q by leveraging Stein's method and a reproducing kernel Hilbert space (e.g., a reproducing kernel space 612). As used herein, the term “reproducing kernel space” can refer to a Hilbert space of functions (e.g., a space where functions, rather than numbers or vectors, serve as the elements) in which evaluation can be performed at any point via an inner product with a specific function from the space, known as a reproducing kernel. In a reproducing kernel space, the kernel can be a symmetric, positive-definite function that defines the structure of the reproducing kernel space.

Specifically, the KSD metric 614 can be represented as the following KSD equation:

$${D}_{s} ( p ‖ q ) := \begin{matrix}max \\ ‖ f ‖ H \leq 1\end{matrix} {E}_{x ∼ p} [ f ( x ) {s}_{q} ( x ) + {∇}_{x} f ( x ) ]$$

$$where$$

$${s}_{q} ( x ) := {∇}_{x} log q ( x ) , H$$

is a reproducing kernel space 612 associated to some positive definite kernel k(⋅,⋅). Indeed, when both p and q have smooth densities, the KSD equation can be expressed more explicitly with the choice of kernel k as the following:

$${D}_{s} ( p ‖ q ) := {E}_{x , {x}^{′} ∼ p} [ {u}_{q} ( x , {x}^{′} ) ]$$

where u_{q}(x,x′) is a Steinized Kernel expressed as follows:

$${{s}_{q} ( x )}^{T} k ( x , {x}^{′} ) {s}_{q} ( {x}^{′} ) + {{s}_{q} ( x )}^{T} {∇}_{{x}^{′}} k ( x , {x}^{′} ) + {∇}_{x} k ( x , {x}^{{′ )}^{T} {s}_{q}} ( {x}^{′} ) + tr ( {∇}_{x , {x}^{′}} k ( x , {x}^{′} ) )$$

Additionally, the multi-perturbation interaction system 100 can approximate Dₛ(p∥q) via samples {Xᵢ} from q as follows:

$${D}_{s} ( p ‖ q ) = \frac{1}{n ( n - 1 )} {\sum }_{1 \leq i \neq j \leq n} {u}_{q} ( {x}_{i} , {x}_{j} )$$

Importantly, when utilizing the above function, the multi-perturbation interaction system 100 only utilizes the score functions and samples from the model distribution q. Indeed, as shown in FIG. 6B, the multi-perturbation interaction system 100 can utilize samples of an observed pairwise representation distribution 616 to generate the latent variable separability metric 620. Accordingly, the multi-perturbation interaction system 100 can determine to utilize samples of the observed pairwise representation distribution 616 or to generate a pairwise score function from the set of observed pairwise perturbation representations. Indeed, when determining to utilize samples of the observed pairwise perturbation representations, the multi-perturbation interaction system 100 can determine the KSD metric 614 without the need for an explicit density estimation.

Additionally, returning to the equation, s(x|δᵢⱼ)=s(x|δᵢ)+s(x|δⱼ)−s(x|δ₀), an FD metric between the left side and the right side of the foregoing equation can be determined using estimated scores of all perturbation groups, i.e.,

$$\int {‖ \hat{s} ( x \vert {δ}_{i j} ) - \hat{s} ( x \vert {δ}_{i} ) - \hat{s} ( x \vert {δ}_{j} ) + \hat{s} ( x \vert {δ}_{0} ) ‖}^{2} p ( x \vert {δ}_{i j} ) d x ,$$

where ŝ denotes estimated score functions obtained from data of a corresponding perturbation group. In some embodiments, score functions can be estimated utilizing a denoising diffusion probabilistic model.

Further, an estimated score function for a double perturbation group can be estimated as

$${\hat{s}}_{i j}^{s e p} := s ( x \vert {δ}_{i} ) + s ( x \vert {δ}_{j} ) - s ( x \vert {δ}_{0} )$$

Accordingly, the multi-perturbation interaction system 100 can determine KSD metrics of single perturbation groups as opposed to quadratic training costs of other methods.

Further, in some embodiments, the multi-perturbation interaction system 100 can utilize an aggregated KSD test to combine multiple KSD metric results evaluated on different choices of kernels. The multi-perturbation interaction system 100 can then utilize a bootstrap method to estimate a p-value for a hypothesis test as follows:

$${H}_{0} : {\hat{s}}_{i j}^{s e p} ( x ) = ∇ log p ( x \vert {δ}_{i j} ) , {H}_{1} : {\hat{s}}_{i j}^{s e p} ( x ) \neq ∇ log p ( x \vert {δ}_{i j} )$$

Indeed, the multi-perturbation interaction system 100 can utilize the foregoing to statistically determine whether a first perturbation and a second perturbation are separable.

More information will now be provided regarding a proof of the previously mentioned equation s(x|δᵢⱼ)=s(x|δᵢ)+s(x|δⱼ)−s(x|δ₀). Specifically, by the injectivity off, the change of variable formula can be applied to express log p(x|T) by:

$$\begin{matrix}log p ( x \vert T ) = log {p}_{Z , U} ( {f}^{- 1} ( x ) \vert T ) + log \vert ( {J}_{f - 1} \vert ( x ) ) \vert \\ {{= log {p}_{Z} ( {f}^{- 1} ( x ) ]}_{Z} \vert T ) + log {p}_{U} ( {f}^{- 1} ( x ) ]}_{U} \vert T ) + log \vert ( {J}_{f - 1} \vert ( x ) ) \vert \\ {{= log {p}_{Z} ( {f}^{- 1} ( x ) ]}_{Z} \vert T ) + log {p}_{U} ( {f}^{- 1} ( x ) ]}_{U} ) + log \vert ( {J}_{f - 1} \vert ( x ) ) \vert\end{matrix}$$

$$where$$

$${J}_{f - 1} ( x ) = \sqrt{det ( {[ \frac{\partial }{\partial x} {f}^{- 1} ( x ) ]}^{T} [ \frac{\partial }{\partial x} {f}^{- 1} ( x ) ] )}$$

if dim(X)>dim(Z)+dim(U);

$${J}_{f - 1} ( x ) = \sqrt{det ( {[ \frac{\partial }{\partial x} {f}^{- 1} ( x ) ] [ \frac{\partial }{\partial x} {f}^{- 1} ( x ) ]}^{T} )}$$

if dim(X)<dim(Z)+dim(U); and

$${J}_{f - 1} ( x ) = \sqrt{det ( [ \frac{\partial }{\partial x} {f}^{- 1} ( x ) ] )}$$

if dim(X) dim(Z)+dim(U). Indeed, the second equality is s by Z⊥U, and the last equality is by T⊥Z.

As previously mentioned, the multi-perturbation interaction system 100 can determine a mean distance of feature embeddings 712 to determine a domain disjointedness measure between a first perturbation and a second perturbation. As shown in FIG. 7, the multi-perturbation interaction system 100 can combine sets of individual perturbation representations to generate a synthetic pairwise perturbation distribution and can utilize the synthetic pairwise perturbation distribution to determine a domain disjointedness measure.

As illustrated in FIG. 7, the multi-perturbation interaction system 100 can generate a first set of individual perturbation representations 704 and a second set of individual perturbation representations 706. For example, the first set of individual perturbation representations 704 can be combined to form a first probability distribution. In particular, as discussed above, the multi-perturbation interaction system 100 can generate representations from a first set of cells exposed to a first perturbation. Moreover, the multi-perturbation interaction system 100 can generate a probability distribution of an aspect of the first set of cells that can be impacted by the first perturbation, such as a first morphology/phenomic appearance or a first transcriptomics count of each cell of the first set of cells. Similarly, the second set of individual perturbation representations 706 can be combined to form a second probability distribution. In particular, the multi-perturbation interaction system 100 can generate representations from a second set of cells exposed to a second perturbation. Further, the multi-perturbation interaction system 100 can generate a probability distribution of an aspect of the second set of cells that can be impacted by the second perturbation, such as a second morphology/phenomic appearance or a second transcriptomics count of each cell of the second set of cells. Further, the multi-perturbation interaction system 100 can additively combine the first set of individual perturbation representations 704 and the second set of individual perturbation representations 706 to generate a synthetic pairwise perturbation distribution 710.

As used herein, the term “synthetic pairwise perturbation distribution” refers to a combination of probability distributions of a first probability distribution of a first perturbation performed on a first set of cells and a second probability distribution of a second perturbation performed on a second set of cells. In other words, the multi-perturbation interaction system 100 determines the synthetic pairwise perturbation distribution by applying a first perturbation to a first set of cells, determining a first probability distribution of the first set of cells after applying the first perturbation, applying a second perturbation to a second set of cells, determining a second probability distribution of the second set of cells after applying the second perturbation, and additively combining the first probability distribution and the second probability distribution.

Indeed, when additively combining the first set of individual perturbation representations 704 and the second set of individual perturbation representations 706 to generate the synthetic pairwise perturbation distribution 710, the multi-perturbation interaction system 100 can determine to account for a control set of representations 708. The control set of representations 708 can be a probability distribution of a control set of cells. For example, the multi-perturbation interaction system 100 can grow or otherwise develop the control set of cells without applying any perturbations to the control set of cells. Responsive to developing the control set of cells, the multi-perturbation interaction system 100 can determine the control set of representations 708 by determining a control probability distribution of an aspect of the control set of cells, such as a cell morphology/phenomic appearance of each cell of the control set of cells or a transcriptomics profile of each cell of the control set of cells.

Specifically, the multi-perturbation interaction system 100 accounts for the control set of representations 708 by determining a first difference between the first set of individual perturbation representations 704 and the control set of representations 708. Additionally, the multi-perturbation interaction system 100 determines a second difference between the second set of individual perturbation representations 706 and the control set of representations 708. The multi-perturbation interaction system 100 combines the first difference and the second difference to generate the synthetic pairwise perturbation distribution 710.

Additionally, the multi-perturbation interaction system 100 can generate an observed pairwise representation distribution 716. As used herein, the term “observed pairwise representation distribution” can refer to a probability distribution of a double perturbation (e.g., a first perturbation and a second perturbation) applied jointly to a set of cells. For example, the multi-perturbation interaction system 100 can apply the first perturbation and the second perturbation to a third set of cells, and determine a third probability distribution from embeddings of the third set of cells after the first perturbation and the second perturbation are applied (e.g., a probability distribution of an aspect of each of the third set of cells that can display impacts of the first perturbation and the second perturbation, such as a morphology/phenomic appearance of each of the third set of cells or a transcriptomics count/profile of each of the third set of cells).

Indeed, in some embodiments, the multi-perturbation interaction system 100 can refine the observed pairwise representation distribution 716 by determining a difference (e.g., a third difference between the observed pairwise representation distribution 716 and the control set of representations 708.

Responsive to determining the synthetic pairwise perturbation distribution 710 and the observed pairwise representation distribution 716, the multi-perturbation interaction system 100 can compare the synthetic pairwise perturbation distribution 710 and the observed pairwise representation distribution to determine a domain disjointedness measure 714 between the first perturbation and the second perturbation. In other words, the multi-perturbation interaction system 100 can compare the synthetic pairwise perturbation distribution 710 and the observed pairwise representation distribution 716 to determine if the first perturbation and the second perturbation are disjoint. Indeed, by utilizing the synthetic pairwise perturbation distribution 710 to represent an artificial combination of effects of the first perturbation and the second perturbation (e.g., combining representations of effects of the first perturbation and the second perturbation applied separately to different sets of cells), and utilizing the observed pairwise representation distribution 716 to represent effects of the first perturbation and the second perturbation when performed jointly on the third set of cells, the multi-perturbation interaction system 100 can determine the domain disjointedness measure 714 of the first perturbation and the second perturbation. In other words, the multi-perturbation interaction system 100 can utilize the domain disjointedness measure 714 to determine a level of composability of the first perturbation and the second perturbation (e.g., the multi-perturbation interaction system 100 utilizes the domain disjointedness measure 714 to determine whether a double perturbation can be additively predicted from effects of the first perturbation or the second perturbation).

For example, similar to equation 310, equation 702 illustrates a test for disjointedness utilized by the multi-perturbation interaction system 100 in accordance with one or more embodiments:

$$p ( x \vert {δ}_{ij} ) - p ( x \vert {δ}_{0} ) = ( p ( x \vert {δ}_{i} ) - p ( x \vert {δ}_{0} ) ) + ( p ( x \vert {δ}_{j} ) - p ( x \vert {δ}_{0} ) )$$

Indeed, to generate the synthetic pairwise perturbation distribution, the multi-perturbation interaction system 100 can account for a control set of representations 708. As shown by the equation 702, the multi-perturbation interaction system 100 accounts for the control set of representations 708 by determining a first difference between the first set of individual perturbation representations 704 and the control set of representations 708. Additionally, the multi-perturbation interaction system 100 determines a second difference between the second set of individual perturbation representations 706 and the control set of representations 708. Further, the multi-perturbation interaction system 100 determines a third difference between the synthetic pairwise perturbation distribution 710 and the control set of representations 708.

Moreover, as shown, the multi-perturbation interaction system 100 can utilize the synthetic pairwise perturbation distribution 710 to determine the domain disjointedness measure 714 between the first set of individual perturbation representations 704 and the second set of individual perturbation representations 706. The multi-perturbation interaction system 100 can utilize the domain disjointedness measure 714 to determine whether perturbations of two non-interacting genes can be separated into distinct features, such that their measures sum. By determining and/or utilizing the domain disjointedness measure 714, the multi-perturbation interaction system 100 can reduce a search space (e.g., a search space for pairwise perturbations) by enabling the multi-perturbation interaction system 100 to predict the outcome of perturbation experiments on cells without actually utilizing perturbations to be performed on cells.

Indeed, for two disjoint perturbations (δᵢ,δⱼ), then

$$\begin{matrix}\text{?} [ h ( x ) \vert {δ}_{i , j} ] - \text{?} [ h ( x ) \vert {δ}_{0} ] = \int h ( x ) [ p ( x \vert {δ}_{l j} ) - p ( x \vert {δ}_{0} ) ] d x \\ = \int h ( x ) [ ( p ( x \vert {δ}_{i} ) - p ( x \vert {δ}_{0} ) + \\ ( p ( x \vert {δ}_{j} ) - p ( x \vert {δ}_{0} ) ) ] d x \\ = \text{?} [ h ( x ) \vert {δ}_{i} ] - \text{?} [ h ( x ) \vert {δ}_{0} ] + \\ \text{?} [ h ( x ) \vert {δ}_{j} ] - \text{?} [ h ( x ) \vert {δ}_{0} ]\end{matrix}$$

The above equation implies that average centered embedding vectors, \vec{h}ᵢ:= [symbol] [h(x)|δᵢ]− [symbol] [h(x)|δ0] and, \vec{h}ⱼ can be defined and utilized to accurately predict \vec{h}_{i,j}=\vec{h}ᵢ+\vec{h}ⱼ without performing perturbation experiments on cells.

Further, the equation 702 can be tested according to the following null hypothesis:

$${H}_{0} : \frac{1}{2} p ( x \vert {δ}_{i j} ) + \frac{1}{2} p ( x \vert {δ}_{0} ) = \frac{1}{2} p ( x \vert {δ}_{i} ) + \frac{1}{2} p ( x \vert {δ}_{j} )$$

With balanced experiments (i.e., p(δ_{O})=p=(δᵢ)=p(δⱼ)=p(δᵢⱼ), samples can be created from the mixture

$$\frac{1}{2} p ( x \vert {δ}_{i j} ) + \frac{1}{2} p ( x \vert {δ}_{0} )$$

by combining data from the controlled and double perturbed groups. Further, in the event of unbalanced experiments, the multi-perturbation interaction system 100 can balance the datasets through downsampling or upsampling.

In addition, it is important to note that when determining the domain disjointedness measure, the multi-perturbation interaction system 100 can model the latent distribution as a finite mixture. In this framework, non-interacting perturbations intervene on different mixing components. Accordingly, in some embodiments, it is appropriate to assume a latent distribution p_{z}(z) admits a form of an L-component mixture accordingly:

$${p}_{z} ( z \vert {T}_{1} , … , {T}_{n} ) = {\sum }_{l = 1}^{L} {w}_{l} \cdot {p}_{{z}_{l}} ( z \vert P a ( {z}_{l} ) )$$

where (w₁ . . . , wL)∈ΔL. In addition, perturbations do not intervene the mixing weights (w₁, . . . w_{L}). Therefore, two perturbations δᵢ, δⱼ are disjoint if Ch(Tᵢ)∩Ch(Tj)=Ø. According to the foregoing, in some embodiments the multi-perturbation interaction system 100 can determine that two perturbations are disjoint according to an additivity in intervened latent distributions, i.e.,

$${p}_{z} ( z \vert {δ}_{i j} ) - {p}_{z} ( z \vert {δ}_{0} ) = ( {p}_{z} ( z \vert {δ}_{i} ) - {p}_{z} ( z \vert {δ}_{0} ) ) + ( {p}_{z} ( z \vert {δ}_{j} ) - {p}_{z} ( z \vert {δ}_{0} ) )$$

As previously mentioned, if a pair of perturbations (e.g., a first perturbation and a second perturbation) are disjoint, they will impact distinct mixing components. Without a loss of generality, if the perturbations, δᵢ and δⱼ intervene with p_{Zli} and p_{Zlj} respectively, then the following can be shown, proving the foregoing:

$${p}_{z} ( z \vert {δ}_{i} ) - {p}_{z} ( z \vert {δ}_{0} ) = {w}_{{l}_{i}} \cdot ( {p}_{{z}_{{l}_{j}}} ( z \vert {δ}_{i} ) - {p}_{{z}_{{l}_{j}}} ( z \vert {δ}_{0} ) )$$

$${p}_{z} ( z \vert {δ}_{j} ) - {p}_{z} ( z \vert {δ}_{0} ) = {w}_{{l}_{j}} \cdot ( {p}_{{z}_{{l}_{j}}} ( z \vert {δ}_{j} ) - {p}_{{z}_{{l}_{j}}} ( z \vert {δ}_{0} ) )$$

$${p}_{z} ( z \vert {δ}_{i j} ) - {p}_{z} ( z \vert {δ}_{0} ) = {w}_{{l}_{i}} \cdot ( {p}_{{z}_{{l}_{j}}} ( z \vert {δ}_{i} ) - {p}_{{z}_{{l}_{j}}} ( z \vert {δ}_{0} ) ) + {w}_{{l}_{j}} \cdot ( {p}_{{z}_{{l}_{j}}} ( z \vert {δ}_{j} ) - {p}_{{z}_{{l}_{j}}} ( z \vert {δ}_{0} ) )$$

The multi-perturbation interaction system 100 can analyze equation 702 and these additional formulations utilizing a variety of different models or metrics. For example, in some implementations, the multi-perturbation interaction system 100 utilizes a mean discrepancy metric. Indeed, as shown in FIG. 7, the multi-perturbation interaction system 100 utilizes a mean distance of feature embeddings 712 between two perturbations as the domain disjointedness measure.

For example, the multi-perturbation interaction system 100 can utilize a synthetic pairwise perturbation distribution to determine a mean discrepancy metric between a first set of individual perturbation representations and a second set of individual perturbation representations. FIG. 8 illustrates the multi-perturbation interaction system 100 mapping a synthetic pairwise perturbation distribution 806 to a reproducing kernel space to determine a mean discrepancy metric.

As shown in FIG. 8, the multi-perturbation interaction system 100 can generate a first set of individual perturbation representations 802 (e.g., from a first set of cells exposed to a first perturbation), a second set of individual perturbation representations 804 (e.g., from a second set of cells exposed to a second perturbation), and an observed pairwise representation distribution 808 (e.g., from a third set of representations of a third set of cells exposed to both the first perturbation and the second perturbation). The multi-perturbation interaction system 100 can additively combine the first set of individual perturbation representations 802 and the second set of individual perturbation representations 804 to generate the synthetic pairwise perturbation distribution 806.

As shown, the multi-perturbation interaction system 100 can map the synthetic pairwise perturbation distribution 806 and the observed pairwise representation distribution 808 to a reproducing kernel space 810. The multi-perturbation interaction system 100 can utilize the reproducing kernel space 810 to determine a mean discrepancy metric between the synthetic pairwise perturbation distribution 806 and the observed pairwise representation distribution 808. Accordingly, the multi-perturbation interaction system 100 can utilize the mean discrepancy metric 812 to determine a domain disjointedness measure between the first set of individual perturbation representations 802 and the second set of individual perturbation representations 804.

More information will now be provided regarding the mean discrepancy metric 812 and how the multi-perturbation interaction system 100 determines the mean discrepancy metric 812. For example, the mean discrepancy metric 812 can be a result of a maximal mean discrepancy (MMD) based two-sample test that compares two distributions based on their embeddings in a reproducing kernel Hilbert space (e.g., a reproducing kernel space 810).

For example, given a feature map φ: X→F_{φ}, where F_{φ} is some Hilbert space (sometimes called the feature space). This feature map φ defines a kernel:

$${k}_{ϕ} : X \times X \to ℝ$$

$${k}_{ϕ} ( x , y ) = 〈 ϕ ( x ) , ϕ ( y ) 〉$$

where [symbol] ⋅, ⋅ [symbol] denotes the inner product of F_{φ}. This definition of kernel k_{φ} induces a space of functions, H_{φ}—from X to R, which is a reproducing kernel Hilbert space (e.g., a reproducing kernel space 810). The reproducing kernel space 810 includes the following reproducing properties:

$$∀ f ∈ {H}_{ϕ} , {〈 f , ϕ ( x ) 〉}_{{H}_{ϕ}} = f ( x )$$

Further, φ(x) can be interpreted as a function of H_{φ}. Further, there exists a special instance of the reproducing property such that:

$$〈 φ ( x ) , φ ( y ) 〉 = {k}_{φ} ( x , y )$$

Moreover, φ(x) can be represented accordingly:

$$ϕ ( x ) : X \to {H}_{ϕ} , ϕ ( x ) ( \cdot ) = {k}_{ϕ} ( x , \cdot ) ∈ {H}_{ϕ}$$

Additionally, the above mentioned definition of H_{φ} (e.g., H_{φ}—from X to R) guarantees that kφ (x, ⋅)∈H_{φ}.

Moreover, in the following,

$${M}_{+}^{1}$$

(X) to denote the space of probability measures over X. Any probability measure can be embedded to the reproducing kernel Hilbert space (e.g., the reproducing kernel space 810). Specifically, the kernel mean embedding of a probability measure P∈H_{φ} can be defined according to the following mapping:

$${μ}_{ϕ} : {M}_{+}^{1} ( X ) \to {H}_{ϕ} , {μ}_{ϕ} ( ℙ ) ( \cdot ) = {\int }_{X} ϕ ( x ) ( \cdot ) ℙ ( d x ) = \text{?} [ ϕ ( X ) ] ( \cdot )$$

where the kernel mean embedding ( [symbol] ) is denoted by μ_{φ}( [symbol] ).

Additionally, for all P,

$$Q ∈ {M}_{+}^{1} ( X ) ,$$

the following assumption applies:

$$M M {D}_{{k}_{ϕ}} ( ℙ , ℚ ) = ‖ \text{?} [ ϕ ( X ) ] - \text{?} [ ϕ ( y ) ] ‖ = ‖ {μ}_{ϕ} ( ℙ ) - {μ}_{ϕ} ( ℚ ) ‖$$

The above-mentioned assumption can be proved thusly:

$${MMD}_{{k}_{ϕ}} ( {𝕄}_{1} , {𝕄}_{2} )$$

$$= sup \text{?} [ f ( X ) ] - \text{?} [ f ( Y ) ]$$

$$f ∈ {H}_{ϕ} : ‖ f ‖ \leq 1$$

$$= sup \text{?} [ f , ϕ ( X ) ] - \text{?} [ 〈 f , ϕ ( Y ) 〉 ]$$

$$f ∈ {H}_{ϕ} : ‖ f ‖ \leq 1$$

$$= sup 〈 f , \text{?} [ ϕ ( X ) ] 〉 - 〈 f , \text{?} [ ϕ ( Y ) ] 〉$$

$$f ∈ {H}_{ϕ} : ‖ f ‖ \leq 1$$

$$= sup 〈 f , \text{?} [ ϕ ( X ) ] - \text{?} [ ϕ ( Y ) ] 〉$$

$$f ∈ {H}_{ϕ} : ‖ f ‖ \leq 1$$

The above proof is completed by observing that the inner product is maximized when:

$$f = \{ \text{?} [ ϕ ( Y ) ] \text{?} [ ϕ ( Y ) ] \} / ‖ \text{?} [ ϕ ( Y ) ] - \text{?} [ ϕ ( Y ) ] ‖$$

Essentially, the above assumption states that the mean discrepancy metric between two distributions is the distance of mean embeddings of features. It also states that MMDₖₚ(P, Q)=0 if and only if μ_{φ}(P)=μ_{φ}(Q)

Further, to be able to utilize the mean discrepancy metric 812 to separate two distributions (e.g., the synthetic pairwise perturbation distribution 806 and the observed pairwise representation distribution 808), the kernel mean embedding can be an injective map, in which case the feature map induces a characteristic kernel. Indeed, k is said to be characteristic on

$${M}_{+}^{1} ( X )$$

if the kernel mean embedding, represented by:

$${μ}_{k} : {M}_{+}^{1} ( X ) \to {H}_{k} , {μ}_{k} ( ℙ ) ( \cdot ) = {\int }_{X} ϕ ( x ) ( \cdot ) ℙ ( d x )$$

is injective. In other words, k is a characteristic kernel if:

$${μ}_{k} ( ℙ , ℚ ) = 0 ⇔ {μ}_{k} ( ℙ ) = {μ}_{k} ( ℚ ) ⇔ ℙ = ℚ$$

Moving forward, the subscript of μ, H is changed from the feature map to the kernel, because work is not done explicitly on the choice of feature maps. Many characteristic kernels may not have tractable feature maps. The above equation states that the mean discrepancy metric 812 is a metric on

$${M}_{+}^{1} ( X )$$

if a characteristic kernel is used.

As previously mentioned, the multi-perturbation interaction system 100 can utilize active learning to predict and/or identify pairwise perturbations. As is described in additional detail below (e.g., FIGS. 9-11), the multi-perturbation interaction system 100 further identifies utilizes prediction failure scores (e.g., the where the sum of the sets of individual perturbation representations is not equal to predicted pairwise representations) to identify candidate perturbation pairs with a high potential for meaningful biological interactions resulting from double perturbations.

As mentioned above, with the measures of biological activity, the multi-perturbation interaction system 100 can further reduce the problem of identifying promising perturbation pairs to active learning. For instance, active learning can include active perturbation interaction matrix completion, uncertainty sampling (e.g., prioritizing selections within a perturbation interaction matrix that skew towards maximizing information gain), density-based methods (e.g., selecting data points that are under-represented or have a high data density to prioritize comprehensively exploring a state space), and expected model change (e.g., generating a prediction for how much a model will change if a particular data point is labeled i.e., tested to obtain actual data).

In one or more embodiments, the multi-perturbation interaction system 100 reduces the problem of identifying promising perturbation pairs to an active-matrix completion problem, which avoids the need for brute-force searches. FIG. 9 illustrates the multi-perturbation interaction system 100 utilizing a pairwise prediction model 902 to select an entry from a perturbation interaction perturbation interaction matrix. As used herein, a “perturbation interaction matrix” refers to an array (e.g., a two-dimensional array arranged in rows and columns) or other digital object for storing information regarding perturbation pairs. For instance, a perturbation interaction matrix can include rows with a set of perturbations and columns with an additional set of perturbations. Moreover, the perturbation interaction matrix includes entries corresponding to a specific pair of perturbations. For example, a specific entry of the perturbation interaction matrix can refer to a measure of biological activity for a specific double gene knockout for a cell.

In one or more embodiments, the multi-perturbation interaction system 100 generates a perturbation interaction matrix for pairs of perturbations. Specifically, the pairs of perturbations can include different pairs of gene knockouts. Furthermore, the perturbation interaction matrix generated by the multi-perturbation interaction system 100 includes pairwise perturbation experiment data plus individual perturbation data that lacks pairwise experimentation data. In other words, the perturbation interaction matrix models the state space for perturbation pairs and includes both entries for measures of biological activity for pairwise perturbations and predictions of measures of biological activities for individual perturbations without pairwise perturbation data. In one or more implementations, the multi-perturbation interaction system 100 has access to all single perturbation distributions p(x|δᵢ) and adaptively selects the pairs i, j on which to collect samples from p(x|δᵢ,δⱼ).

As shown in FIG. 9, the multi-perturbation interaction system 100 receives sets of individual perturbations 900 (and/or pairwise perturbations from already conducted experiments). As used herein, the “pairwise prediction model” refers to a model that the multi-perturbation interaction system 100 utilizes to select an entry from the perturbation interaction matrix. Specifically, the pairwise prediction model 902 generates a plurality of predicted pairwise perturbation interaction scores for entries of the perturbation interaction matrix and balances that with corresponding information gain prediction scores to perform an active-matrix completion technique (e.g., which selects an entry from the perturbation interaction matrix).

As used herein a “predicted pairwise perturbation interaction score” refers to a prediction for a perturbation pair (e.g., in the perturbation interaction matrix) regarding a perturbation interaction score. In other words, the multi-perturbation interaction system 100 generates a prediction of reward for a perturbation pair, where the multi-perturbation interaction system 100 only has access to single perturbation data for that perturbation pair. For instance, the multi-perturbation interaction system 100 has access to a plurality of individual perturbation representations (e.g., including the perturbation pair), but does not have data for exposing a cell to a specific set of double perturbations. As such, the multi-perturbation interaction system 100 utilizes the pairwise prediction model to generate the predicted pairwise perturbation interaction score.

As used herein, an “information gain prediction score” refers to an indication of the amount of increase in knowledge or reduction in uncertainty. Specifically, the multi-perturbation interaction system 100 generates an information gain prediction score based on an entropy measure, represented as a probability distribution. For example, the multi-perturbation interaction system 100 references existing knowledge (e.g., the plurality of pairwise perturbations and the corresponding measures of biological activities) to determine the information gain prediction score for a particular perturbation pair. To illustrate, a higher information gain prediction score indicates that if selected, the entry is more informative and valuable for reducing uncertainty.

As shown in FIG. 9, based on an information gain prediction score 903 (e.g., a corresponding information gain prediction) and a predicted pairwise perturbation interaction score 905, the multi-perturbation interaction system 100 utilizes an active-matrix completion algorithm 904 to select an entry 906. As used herein, the term “active-matrix completion algorithm” refers to actively selecting and acquiring certain entries of an incomplete matrix to efficiently fill in or predict the missing values. Specifically, the perturbation interaction matrix includes as entries a plurality of pairwise perturbations and corresponding measures of biological activities (e.g., already performed double perturbations) and the incomplete entries correspond with perturbation pairs without pairwise perturbations already performed (e.g., the multi-perturbation interaction system 100 only has access to individual perturbation data). Further, the perturbation interaction matrix can include latent variable separability measures (e.g., that include latent variable separability measure as discussed above in FIGS. 1-7). In addition, the perturbation interaction matrix can include a plurality of domain disjointedness measures (e.g., that include the domain disjointedness measure as discussed above in FIG. 1 and FIGS. 8-9). The perturbation interaction matrix can also include a combination (e.g., a weighted combination) of the latent variable separability measures and the domain disjointedness measure). As mentioned, the multi-perturbation interaction system 100 utilizes the active-matrix completion algorithm 904 to select one or more incomplete entries based on a balance between the information gain prediction score 903 and reward (e.g., the predicted pairwise perturbation interaction score 905).

As illustrated, the multi-perturbation interaction system 100 utilizes the active-matrix completion algorithm 904 to select the entry 906 from the perturbation interaction matrix. As used herein, the term “perturbation pair” refers to a pair of perturbations (e.g., gene knockouts, compounds, or a gene knockout-compound pair) corresponding to an entry of the perturbation interaction matrix. Specifically, the multi-perturbation interaction system 100 has not performed experimentation of exposing a cell to the perturbation pair corresponding to the perturbation interaction matrix entry.

In some embodiments, the multi-perturbation interaction system 100 can transmit a perturbation pair to initiate additional experimental processes for generating additional measures of biological activity, such as a latent variable separability measure or a domain disjointedness measure. Responsive to determining an additional measure of biological activity (e.g., an additional latent variable separability measure or an additional domain disjointedness measure), the multi-perturbation interaction system 100 can update the perturbation interaction matrix based on the additional measure of biological activity.

The following description provides additional details of efficiently discovering interacting perturbation pairs (e.g., gene pairs). As discussed above, the multi-perturbation interaction system 100 utilizes the measure of biological activity (e.g., the prediction error or loss) as an indicator of potential perturbation pair interactions (e.g., gene interactions). In some embodiments, the multi-perturbation interaction system 100 identifies gene-pair knockouts that induce large interactions (e.g., a large measure of biological activity) in order to discover additional gene-gene relationships within the constraints of only having a fixed number of experimental resources (e.g., performing double gene knockouts on all pairs is unfeasible).

As alluded to above, in one or more implementations the multi-perturbation interaction system 100 reduces the problem of discovering additional interacting gene pairs efficiently to a matrix completion framework. For instance, the multi-perturbation interaction system 100 utilizes X to denote the space of possible experiment designs, which is a set of tuples G×G where G is the set of genes, each associated with a reward score R: X→ [symbol] . For instance, the reward for pair (i,j)∈E×G is defined as follows:

$$R ( i , j ) := {( \text{?} [ {h}_{i , j} - {h}_{i} - {h}_{j} ] )}^{2} where {h}_{i} := E [ h ( Y ) \vert {x}_{i} - 1 ] - E [ h ( Y ) \vert x = 0 ]$$

In the above notation, the perturbation interaction matrix is symmetric as the reward function is invariant to the order of the perturbations.

Specifically, the multi-perturbation interaction system 100 utilizes a framework of adaptive sampling for discovery with information directed sampling. For instance, adaptive sampling for discovery refers to a sequential decision-making problem that chooses entries/points that yield information to improve a model estimate. Furthermore, information directed sampling refers to an optimization approach that balances exploration and exploitation while learning from partial feedback. To illustrate, the multi-perturbation interaction system 100 implements the methods described in Ziping xu, Eunjae shim, Ambuj Tewari, and Paul Zimmerman, Adaptive Sampling for Discovery, arXiv:2205.14829v3, 2022 and Daniel Russo and Benjamin Van Roy, Learning to Optimize Via Information-Directed Sampling, arXiv:1403.5556v7, 2017, which are both fully incorporated by reference herein.

To provide more information regarding information directed sampling, let A denote the action space of possible experiment designs, which in this case is the set of perturbations

$$Δ := \{ {δ}_{i j} : i , j ∈ n , i > j \}$$

where n is a total number of distinct perturbations. To simplify the foregoing notation, the perturbation pair i,j selected at step k is denoted as a^{(k)}:=(i^{(k)},j^{(k)})∈Δ. D(Δ) can be a set of possible (categorical) distributions defined over Δ. Each time an action, a^{(k)} is selected, a corresponding element of an (unknown) reward matrix, R, is revealed to an agent. Further, let

$${H}_{t} = {( ( {a}^{( k )} , {R}_{{i}^{( k )} , {j}^{( k )}} ) )}_{k = 1}^{t - 1}$$

denote a history of actions and their corresponding rewards until round t, and Δₜ denotes a set of remaining actions at round t; i.e., the pairs of perturbations that have not yet been tested experimentally. Further, a policy π is defined as a map from Hₜ to D(Δₜ). An multi-perturbation interaction system 100 policy, π_{IDS} maintains a posterior distribution over R given the data observed up to round t, which can be denoted as p(R|Hₜ).

Further, a sub-optimality of an action with respect to a set of premises can be described by comparing a reward of a given action to a reward of a best (e.g., most optimal) action that could have been selected at time t, under the agent's current posterior over the reward matrix. Indeed, this can be evaluated by sampling a plausible reward matrix from the posterior \dot{R}~(R|Hₜ) and then comparing the reward from an action a to the reward the agent could have received from selecting the most optimal action, a*=arg maxa_{∈Δt}\dot{R}(a), where \dot{R}(a^{(k)}):=\dot{R}ᵢ_{^{(k)}}_{,j}_{^{(k)}} for a^{(k)}:=(i^{(k)},j^{(k)}). This can be referred to as an expected instantaneous regret incurred by an action and can be defined as: Δₜ(a)= [symbol] _{\dot{R}~p(R|H}_{ₜ}₎[\dot{R}(a*)−\dot{R}(z)]. Additionally, an information gain about a top T−t+1 remaining actions can be defined as follows:

$$g ( a ) = MI ( {a}_{t , 1}^{*} , … , {a}_{t , T - t + 1}^{*} ; R ( a ) \vert {H}_{t} , {δ}_{t} = a )$$

In one or more embodiments, the multi-perturbation interaction system 100 formalizes a problem of selecting an entry from a matrix as a sequential Bayesian optimal experimental design. For example, the experiments x∈X is designed with outcomes y∈Y governed by a generative process y~p(y|γ,x) with parameters γ. In some embodiments, the experiments are performed sequentially (x₁ . . . , x_{T}) with the objective of maximizing a measure of utility, e.g., the information gain. In other words, the multi-perturbation interaction system 100 iteratively selects entries from a matrix that indicate the highest potential information gain. For instance, the multi-perturbation interaction system 100 represents the Bayesian optimal experimental design as:

$${x}_{t}^{*} = \begin{matrix}arg max MI ( γ , \{ x , y \} \vert {D}^{t} ) \\ x ∈ \{ X ∖ {D}_{x}^{t}\end{matrix}$$

In the above notation,

$${x}_{t}^{*}$$

denotes an optimal solution for running an experiment from the set of entries, which is equivalent to an arg max operation that finds the input that maximizes a function of maximizing the mutual information between an experimental outcome and a parameter of interest (e.g., parameters γ). Further, the above notation indicates that x is an element of the set of elements that are in set X but not in set

$${D}_{x}^{t} ,$$

where D^{t} indicates a dataset of observed data (e.g., the plurality of pairwise perturbations and the corresponding measures of biological activity).

In some embodiments, the multi-perturbation interaction system 100 solves an optimization problem (of acquiring the most information in an efficient manner) by estimating a posterior probability p(γ|Dᵢ) and knowledge of the generative process p(y|γ,x) and nested integral over y and γ which can suffer from poor convergence rates when estimated from samples. Accordingly, the multi-perturbation interaction system 100 utilizes the above methods to search over a combinatorial space of sets of experiments (e.g., perturbation pairs) to be selected at each step.

In one or more embodiments, the multi-perturbation interaction system 100 further implements a multi-armed bandits framework. For instance, the multi-armed bandits framework includes learning a policy I which maps a history of observations (e.g., the measures of biological activities for the comparison between pairwise representations and predicted pairwise representations) to a distribution over a set of possible actions (A), where each action a∈A is associated with an unknown potentially stochastic reward ƒ(a), such that after T actions sampled from the policy (a₁ . . . , a_{T}) the regret

$${R}_{T} = T {max}_{a ∈ A} \text{?} [ f ( a ) ] - {\sum }_{i}^{T} f ( {a}_{i} )$$

is minimized. In other words, the regret bounds for bandit optimization are characterized in terms of information gain.

In one or more embodiments, the multi-perturbation interaction system 100 denotes the set of possible distributions defined over X by D(X), the history of actions and their corresponding rewards until round t by

$${H}_{t} = {( ( {x}_{k} , R ( {x}_{k} ) ) )}_{k = 1}^{t - 1}$$

and Xₜ denotes the set of available designs at round t. For instance, the policy Π_{IDS} (e.g., information directed sampling policy) is defined as a map from Hₜ to D(Xₜ). Further, for a discrete space of actions, each element of D(Xₜ) is a vector which represents the distribution over available actions. Specifically, the multi-perturbation interaction system 100 defines an instant regret of taking an action as Δₜ(x)= [symbol] _{ƒ~p(R|H}_{ₜ}₎[max_{x′∈x}_{ₜ}ƒ(x′)−ƒ(x)], where the expectation is over samples from the posterior over the reward function ƒ given the history of observations Hₜ. Additionally, the multi-perturbation interaction system 100 defines

$$g ( x ) = MI ( {X}_{t , 1}^{*} , … , {X}_{t , T - t + I}^{*} ; R ( {X}_{t} ) \vert {H}_{t} , {X}_{t} = x )$$

as the information gain about the top T−t+1 unselected actions. Further the information directed sampling policy at round t can be computed by minimizing the information ratio:

$${π}_{IDS} {∈}^{arg min {Ψ}_{π , t} := \frac{{( {Δ}_{t}^{T} π )}^{λ}}{{g}_{t}^{T} π}}$$

$$π ∈ D ( {X}_{t} )$$

In the above notation, λ controls the tradeoff between lower instant regret (exploitation) and higher information gain (exploration). For instance, the multi-perturbation interaction system 100 utilizes an approximate algorithmic choice, replacing gₜ with a conditional variance

$${v}_{t} ( x ) = {Var}_{t} ( \text{?} [ R ( {X}_{t} ) \vert {X}_{1}^{*} , {X}_{t} = x ] ) ,$$

as it is lower bound on the information gain gₜ(x)≥vₜ(x). Furthermore, the multi-perturbation interaction system 100 adopts low-rank matrix with a prior of row and column spaces being sampled from a standard Gaussian. In particular, to obtain samples from the posterior distribution over the low-rank (where m is the rank) reward matrix, the multi-perturbation interaction system 100 utilizes a stochastic variational inference.

In some embodiments, the multi-perturbation interaction system 100 utilizes the following algorithm for adaptive sampling for discovery for designing gene pair knockouts:

1: Initialize H₁ = { }, batch size b
2: for t = 1...., T do
3: Estimate posterior p(R| Hₜ)
4: Compute information ratio Ψₜ
5: Pick batch (a₁,...,a_{b}) greedily which minimize Ψₜ
6: Perform experiments and compute {Rₐ₁, . . ., R_{ab}}
7: Update Hₜ₊₁ = Hₜ ∪ {(a₁, Rₐ₁, . . ., (a_{b}, Rₐ₁)}
8: end for

For instance, the above algorithm indicates that for adaptive sampling for discovery, the multi-perturbation interaction system 100 initializes the history of observations for a first perturbation pair to T (e.g., the last perturbation pair observed) and sequentially from 1 to T, the multi-perturbation interaction system 100 estimates a posterior distribution (e.g., which indicates a posterior probability of the reward given the specific instance of observation). Furthermore, the multi-perturbation interaction system 100 computes the information ratio for the specific instance (e.g., t . . . T) and picks a batch from (a₁, . . . , a_{b}), which indicates the available perturbation pairs for selection. Moreover, as indicated, the multi-perturbation interaction system 100 performs experiments for the selected perturbation pair and computes the actual measure of biological activity. From the computed actual measure of biological activity, the multi-perturbation interaction system 100 can update the perturbation interaction matrix (or the history of observations).

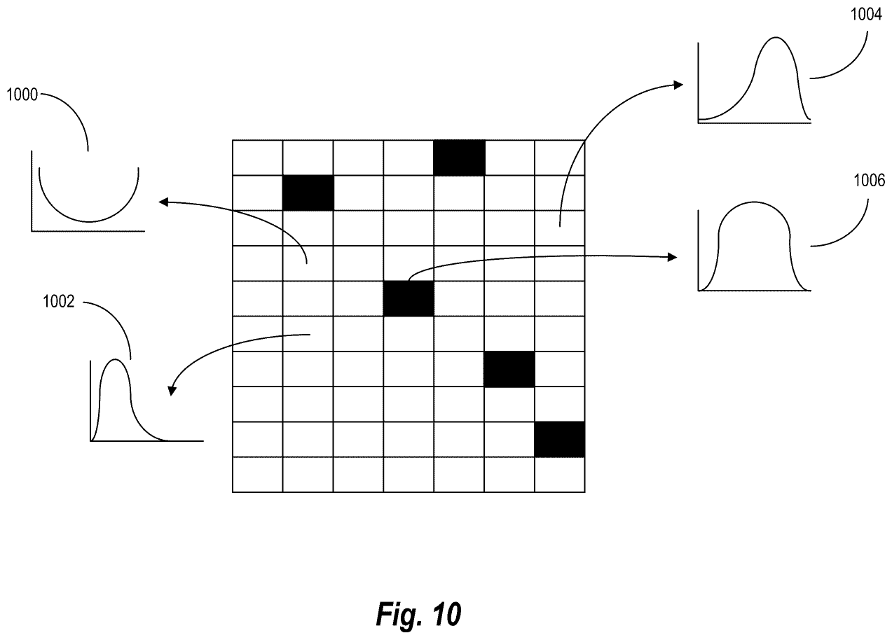

FIG. 10 illustrates a matrix with various entries and corresponding probability distributions treated as reward in accordance with one or more embodiments. For example, FIG. 10 shows a first entry with a first probability distribution 1000, a second entry with a second probability distribution 1002, a third entry with a third probability distribution 1004, and a fourth entry with a fourth probability distribution 1006. For instance, FIG. 10 shows the fourth entry as filled in (e.g., shaded), which indicates that it is for a perturbation pair where a pairwise perturbation experiment has already been performed.

As shown in FIG. 10, the various probability distributions represent a posterior over an error. Specifically, the posterior refers to a posterior distribution (e.g., Bayesian) that represents the updated probability about a predicted pairwise perturbation interaction score in view of observed data (e.g., the pairwise perturbation interaction score which indicates the measure of biological activity). In other words, FIG. 10 illustrates a probabilistic estimate of potential errors (e.g., the predicted pairwise perturbation interaction scores) for the plurality of perturbation pairs (e.g., as predicted by the pairwise prediction model). Accordingly, FIG. 10 shows the probability distributions, which provides a distribution of possible interaction scores (e.g., reward) and represents the uncertainty and reliability of the prediction (e.g., the information gain).

To illustrate, FIG. 10 shows that the first probability distribution 1000 indicates uncertainty in the reliability of a prediction, namely that a selection of the entry corresponding to the first probability distribution 1000 could be high error (e.g., a meaningful interaction indicated by the large scalar difference between the combination of the individual representations and the pairwise perturbation representation) or a low error (e.g., an interaction that does not deviate from the normal assumptions of combining individual perturbations). Further, the second probability distribution 1002 shows low error at a probability below 0.5, the third probability distribution 1004 shows high error at a probability above 0.5, and the fourth probability distribution shows a high error (e.g., at approximately 0.5).

Moreover, the multi-perturbation interaction system 100 utilizes the pairwise prediction model to generate the probability distribution shown for each entry of the perturbation interaction matrix, where the probability distribution indicates reward (e.g., by the error) and information gain (e.g., via the uncertainty or reliability of the prediction). Furthermore, the multi-perturbation interaction system 100 utilizes the active-matrix completion algorithm to select an entry from the perturbation interaction matrix for initiating downstream experimentation. Thus, for instance, the multi-perturbation interaction system 100 can select the entry corresponding to the first probability distribution 1000 (e.g., utilizing the active-matrix completion algorithm) due to the high information gain potential (e.g., uncertainty as to whether the perturbation pair is high reward or low reward).

As mentioned above, the multi-perturbation interaction system 100 can select an entry from the perturbation interaction matrix that corresponds to a perturbation pair and initiate one or more additional downstream experiments for the perturbation pair. FIG. 10 illustrates the multi-perturbation interaction system 100 initiating downstream experimentation and generating an additional measure of biological activity in accordance with one or more embodiments.

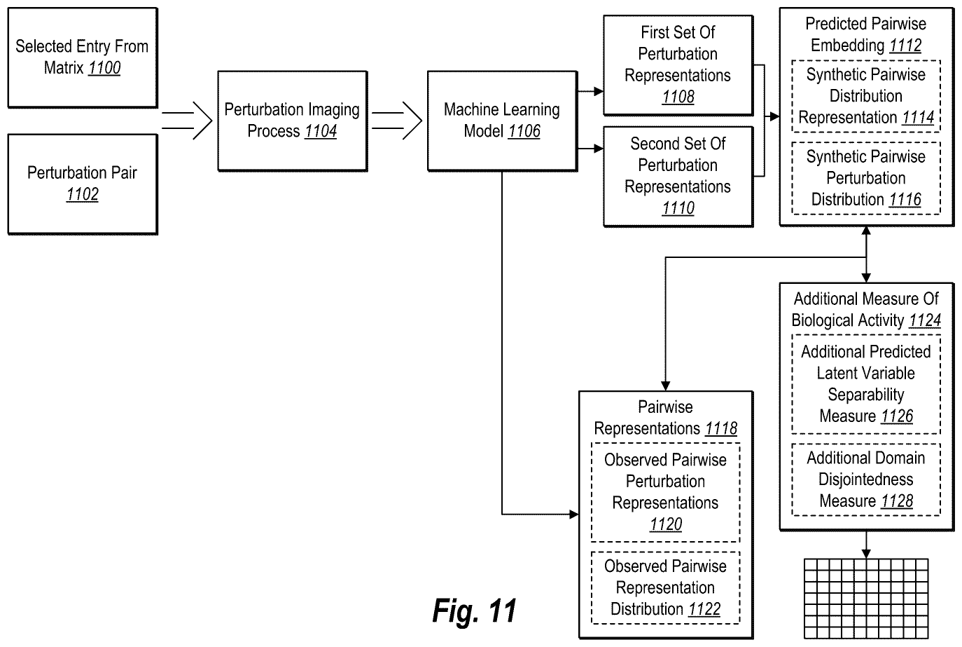

FIG. 11 shows the multi-perturbation interaction system 100 transmitting a perturbation pair 1102 corresponding to an entry 1100 from the perturbation interaction matrix to initiate additional experimental processes for generating an additional measure of biological activity 1124 for the perturbation pair. Specifically, FIG. 11 shows the multi-perturbation interaction system 100 performing a perturbation imaging process 1104 (e.g., as discussed in FIG. 2) on the perturbation pair 1102. For instance, the perturbation imaging process 1104 for the perturbation pair 1102 includes exposing a cell to the perturbation pair 1102 (e.g., a specific double gene knockout as indicated by the entry in the perturbation interaction matrix) and imaging the cell exposed to the perturbation pair 1102. Furthermore, as shown, the multi-perturbation interaction system 100 utilizes a machine learning model 1106 to process the images of the cell exposed to the perturbation pair 1102 to generate various embeddings.

As shown, the multi-perturbation interaction system 100 utilizes the machine learning model 1106 to generate a first individual perturbation first set of perturbation representations 1108 corresponding to a perturbation from the perturbation pair 1102 and a second individual perturbation second set of perturbation representations 1110 corresponding to a second perturbation from the perturbation pair 1102. For instance, the multi-perturbation interaction system 100 generates the individual perturbation representations from exposing cells to the perturbations individually (e.g., not in combination). Moreover, as shown, the multi-perturbation interaction system 100 generates a predicted pairwise predicted pairwise representation 1112 from combining the first individual perturbation first set of perturbation representations 1108 and the second individual perturbation second set of perturbation representations 1110. The predicted pairwise representation can be a synthetic pairwise distribution representation 1114 or a synthetic pairwise perturbation distribution 1116

Additionally, the multi-perturbation interaction system 100 generates a pairwise representation 1118 from a cell being exposed to the perturbation pair 1102. In some embodiments, the pairwise representation 1118 can be an observed pairwise perturbation representation 1120. In some embodiments, the pairwise representation 1118 can be an observed pairwise representation distribution 1122. As shown, the multi-perturbation interaction system 100 compares the pairwise representation 1118 (e.g., the actual representation resulting from exposing a cell to the perturbation pair 1102) with the predicted pairwise predicted pairwise representation 1112 (e.g., the predicted representation resulting from individually exposing cells to perturbations). From the comparison, the multi-perturbation interaction system 100 generates an additional pairwise perturbation interaction score which indicates the additional measure of biological activity 1124. In some embodiments, the additional measure of biological activity 1124 can be an additional latent variable separability measure 1126. In some embodiments, the additional measure of biological activity can be an additional domain disjointedness measure 1128.

As further shown, the multi-perturbation interaction system 100 utilizes the additional measure of biological activity 1124 and updates the perturbation interaction matrix (e.g., discussed in FIGS. 3-4) to include the additional measure of biological activity 1124 for the perturbation pair 1102. As discussed above in FIGS. 9 and 10, the multi-perturbation interaction system 100 selects an entry from the perturbation interaction matrix that corresponds to a perturbation pair, based on a balance between information gain and the reward (e.g., the predicted pairwise perturbation interaction score). Thus, FIG. 11 illustrates the multi-perturbation interaction system 100 further testing the entry to determine the actual measure of biological activity (e.g., the distance between the pairwise representation and the predicted pairwise representation). Accordingly, the multi-perturbation interaction system 100 updates the perturbation interaction matrix to include the actual measure of biological activity.

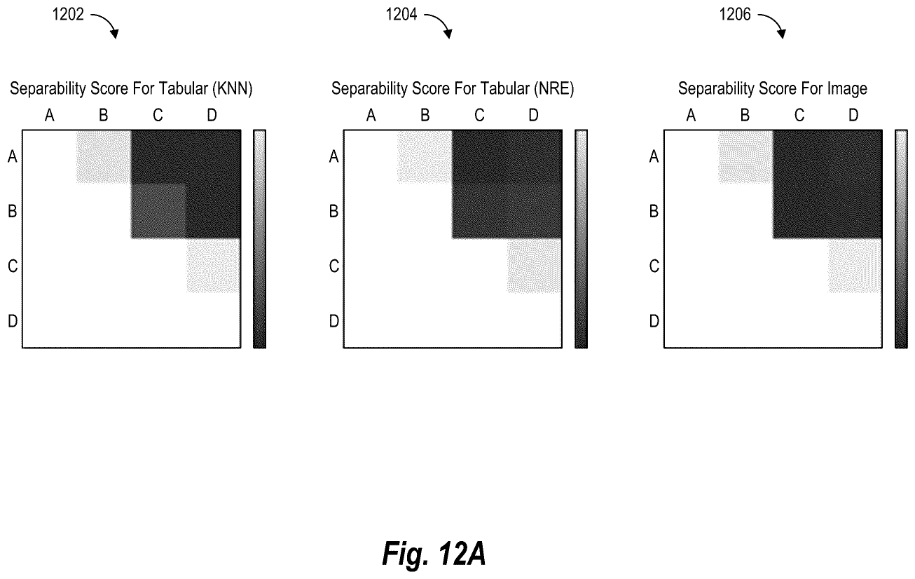

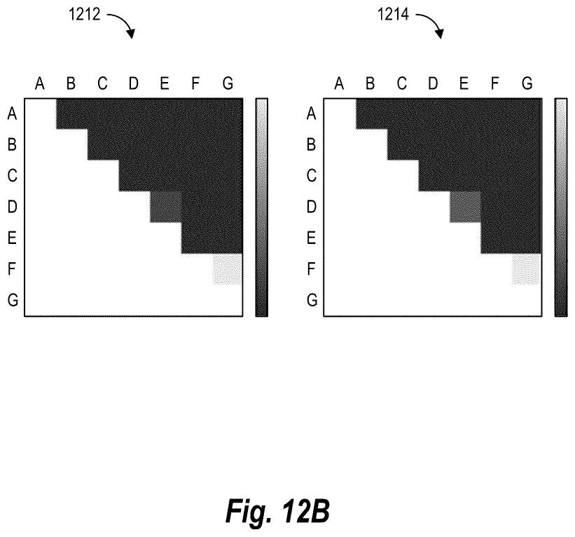

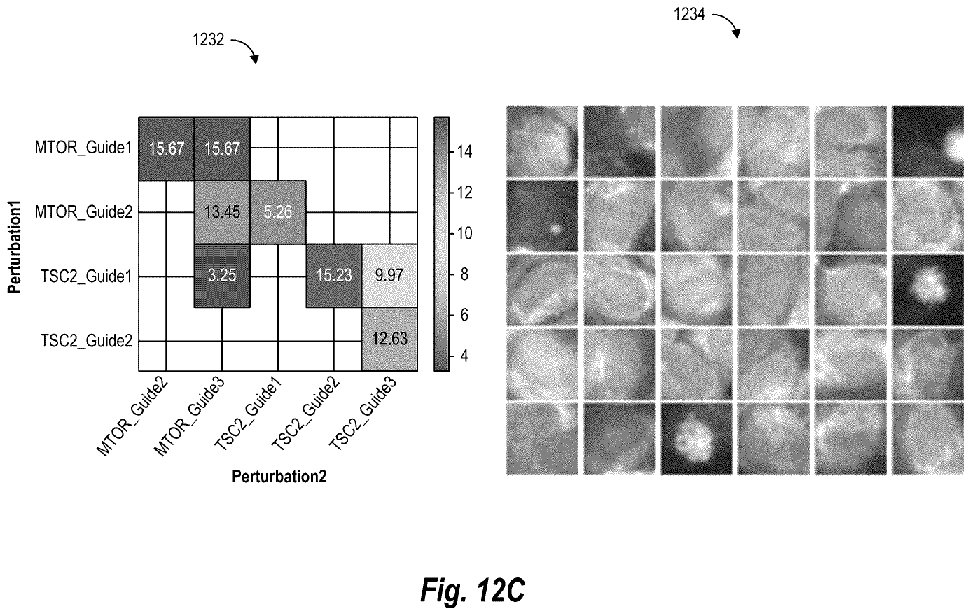

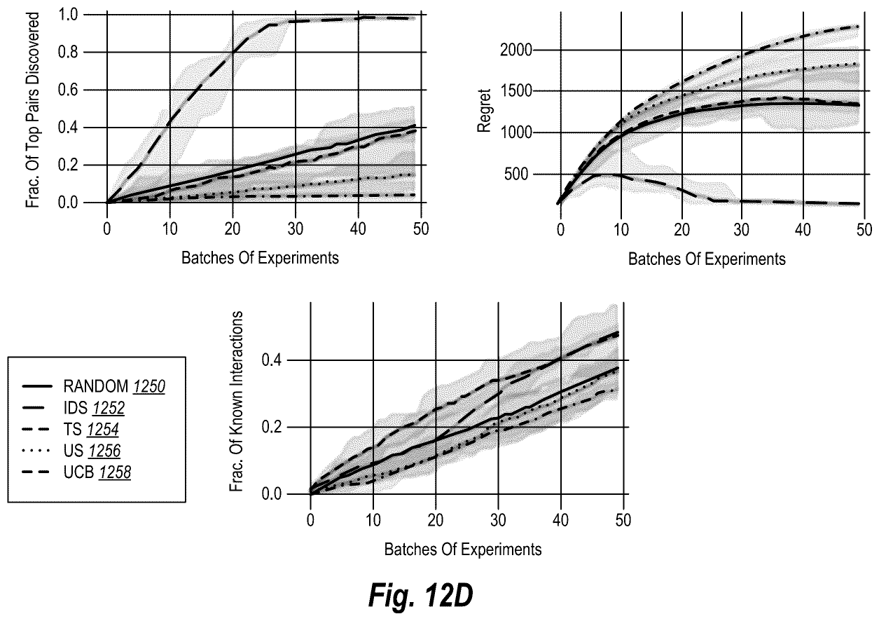

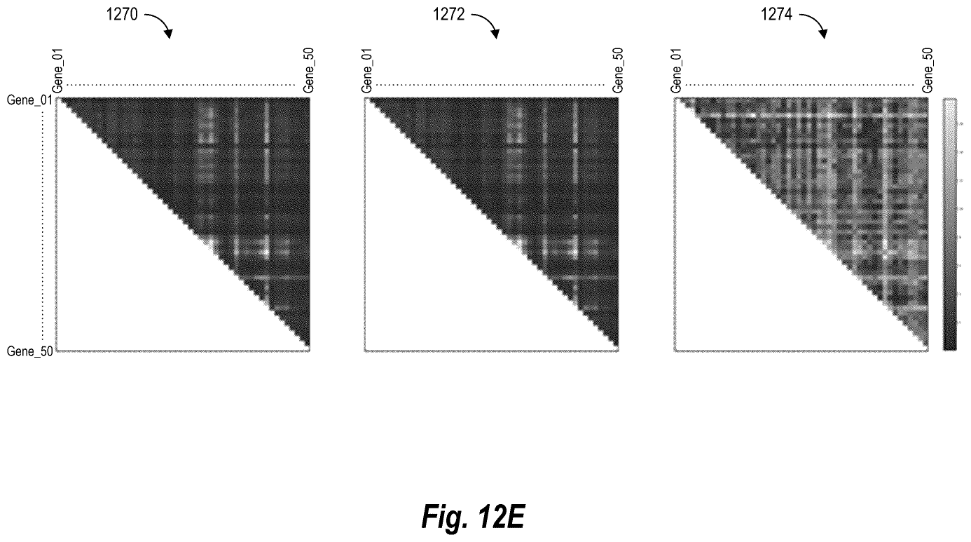

In one or more embodiments, after updating the perturbation interaction matrix to include the additional measure of biological activity 1124, the multi-perturbation interaction system 100 further performs the acts and processes discussed above in FIGS. 9-10. Specifically, the multi-perturbation interaction system 100 utilizes the pairwise prediction model to generate the predicted pairwise perturbation interaction scores and the information gain prediction scores based on the updated matrix. For example, the multi-perturbation interaction system 100 utilizes the active-matrix completion algorithm to further select an additional entry from the perturbation interaction matrix to initiate downstream experimentation with the same principles discussed in FIGS. 12A-E. Accordingly, the multi-perturbation interaction system 100 iteratively updates the perturbation interaction matrix, generates updated predictions, and initiates downstream experimentation in an efficient manner to explore the state space (e.g., without performing brute force searches).

Further information regarding one or more embodiments of the multi-perturbation interaction system 100 utilized to perform active learning approaches described above with regard to FIGS. 9-11 will now be provided. For example, to obtain samples from a posterior distribution of a low-rank reward matrix (with rank m), the multi-perturbation interaction system 100 can utilize stochastic variational inference. The multi-perturbation interaction system 100 trains a variational posterior for 5000 epochs with a learning rate of 0.01 utilizing an ADAM optimizer. Additionally, the multi-perturbation interaction system 100 generates k samples from the posterior. For each algorithm, the multi-perturbation interaction system 100 performs a sweep over a set of hyperparameters. According to the sweep, the multi-perturbation interaction system 100 determines one or more hyperparameters from the set of hyperparameters to perform an experiment over 10 different seeds to generate final results. For example, in an example implementation the multi-perturbation interaction system 100 determined m∈{3,5,7,10,12} and k∈{500, 750, 1000, 1500}. Additionally, in another example implementation the multi-perturbation interaction system 100 determined m∈{3, 5, 7, 10, 12} and k∈{500, 750, 1000, 1500}. Further, in an additional implementation the multi-perturbation interaction system 100 determined m∈{3, 5, 7, 10, 12}, β∈{0.01, 0.1, 0.2, 0.5, 1, 2, 5} and k∈{500, 750, 1000, 1500}, where the multi-perturbation interaction system 100 utilized 3 to control the exploration.

Additional information will now be provided in FIGS. 12A-12E regarding experimental results achieved by one or more implementations of the multi-perturbation interaction system 100.

As previously alluded to, the multi-perturbation interaction system 100 can determine a measure of biological activity of a pairwise perturbation by determining a measure of latent variable separability between a first perturbation and a second perturbation. FIG. 12A illustrates experimental results 1202-1206 (specifically, latent variable separability measures) determined by an experimental embodiment of the multi-perturbation interaction system 100. For all three experimental results (e.g., a first experimental result 1202, a second experimental result 1204, and a third experimental result 1206), lighter colors indicate stronger interactions. Ground truth interacting pairs for all three experiments were A-B and C-D.

As shown, FIG. 12A depicts the first experimental result 1202, the second experimental result 1204, and the third experimental result 1206. The first experimental result 1202 depicts latent variable separability measures determined from synthetic tabular data utilizing a k-Nearest Neighbor (KNN) based Kullback-Leibler estimator. As shown, utilizing the KNN-based KL estimator, the experimental embodiment of the multi-perturbation interaction system 100 correctly identified the ground truth interacting pairs of A-B and C-D. The second experimental result 1204 depicts latent variable separability measures determined from synthetic tabular data utilizing an NRE-based KL estimator. As shown, utilizing the NRE-based KL estimator, the experimental embodiment of the multi-perturbation interaction system 100 correctly identified the ground truth interacting pairs of A-B and C-D. The third experimental result 1206 depicts latent variable separability measures determine from synthetic images. As shown, the experimental embodiment of the multi-perturbation interaction system 100 correctly identified the ground truth interacting pairs of A-B and C-D from the synthetic images.

As previously mentioned, the multi-perturbation interaction system 100 can determine a measure of biological activity for a pairwise perturbation by determining a domain disjointedness measure for a first perturbation and a second perturbation of the pairwise perturbation. FIG. 12B illustrates experimental results 1212-1214 (specifically, domain disjointedness measures) determined by an experimental embodiment of the multi-perturbation interaction system 100. For both experimental results (e.g., a first experimental result 1212 and a second experimental result 1214), lighter colors indicate stronger interactions. The ground truth interaction pairs for both experiments were D-E and F-G.

As shown, FIG. 12B depicts the first experimental result 1212 and the second experimental result 1214. The first experimental result 1212 depicts domain disjointedness measures determined from synthetic tabular data utilizing MMD-based statistics with a Matern 2.5 kernel. As shown, utilizing the Matern 2.5 kernel, the experimental embodiment of the multi-perturbation interaction system 100 correctly identified the ground truth interacting pairs of D-E and F-G. The second experimental result 1214 depicts domain disjointedness measures determined from synthetic tabular data utilizing MMD-based statistics with an RBF kernel. As shown, utilizing the RBF kernel, the experimental embodiment of the multi-perturbation interaction system 100 correctly identified the ground truth interacting pairs of D-E and F-G.

As previously mentioned, the multi-perturbation interaction system 100 improves the accuracy and operational flexibility of implementing systems. FIG. 12C illustrates experimental results achieved by an experimental embodiment of the multi-perturbation interaction system 100. Specifically, FIG. 12C illustrates a graph 1232 (left) the experimental embodiment of the multi-perturbation interaction system 100 determining latent variable separability metrics between different CRISPR guides of two genes, TSC2 and MTOR. In the graph 1232, missing pairs indicates that corresponding pairwise data was not collected for the corresponding pairwise perturbation. The squares with higher latent variable separability metrics indicate that the same gene was targeted by the experiments, whereas the squares with lower latent variable separability metrics indicate that the experiments targeted different genes.

Additionally, FIG. 12C shows a collection of images of cells 1234. Specifically, the collection of images of cells 1234 are random samples of actual single cell images used in the experiments that generated the data displayed in the graph 1232. Notably, visual analysis of the cells 1234 provide very little distinguishing information. Indeed, as discussed previously, despite the excessive computational resources and time required by conventional systems to perform assays, such assays are difficult to analyze and extract useful information regarding perturbation pairs. As illustrated in FIG. 12C, embodiments of the multi-perturbation interaction system 100 can efficiently generate accurate interaction metrics between perturbations from unstructured data resulting from experimental assays.

As mentioned above, the multi-perturbation interaction system 100 more efficiently searches the state space as compared to other approaches, without having to resort to brute-force searching techniques. FIG. 12D illustrates an example implementation of the multi-perturbation interaction system 100 outperforming other approaches in discovering perturbation pairs with meaningful interactions (e.g., interactions beyond that expected from individual perturbations) utilizing a new benchmark task for in-silico validation. In one or more embodiments, experimenters tested the multi-perturbation interaction system 100 by collecting a benchmark dataset of all pairs of gene knockouts for 50 genes in HUVEC cells (e.g., human umbilical vein endothelial cells) with three CRISPR guides per gene (e.g., clustered regularly interspaced short palindromic repeats, which allows experimenters to make precise changes to DNA).

For instance, experimenters ran experiments with embeddings from a DenseNet-based classifier described in G. Huang, Z. Liu, L. Van Der Maaten, and K. Q. Weinberger, Densely connected convolutional networks, in Proceedings of the IEEE conference on computer vision and pattern recognition, pages 4700-4708, 2017. The experimenters train the DenseNet-based classifier on an rxrxl dataset described in M. Sypetkowski, M. Rezanejad, S. Saberian, O. Kraus, J. Urbanik, J. Taylor, B. Mabey, M. Victors, J. Yosinski, A. R. Sereshkeh, et al., Rxrx1: A dataset for evaluating experimental batch correction methods, in Proceedings of the IEEE/CVF Conference on Computer Vision and Pattern Recognition, pages 4284-4293, 2023. In some embodiments, the experimenters average the generated embeddings across all guides and replicates, resulting in 1225 pairwise gene embeddings which form an ‘unknown’ target h_{i,j} and 50 single gene embeddings hᵢ, which are assumed to be known at the start of an active learning experiment.

As shown in FIG. 12D, the experimenters measure an experimental implementation of the multi-perturbation interaction system 100 (e.g., IDS 1252) against random 1250 (e.g., a random policy of selection), TS 1254 (e.g., Thompson Sampling, which includes an agent maintaining a probability distribution over expected rewards for each action such that the agent at each step samples from these distributions and selects the action with the highest sampled reward), US 1256 (e.g., uncertainty sampling, which includes an algorithm to select instances with high uncertainty, in other words uncertainty sampling skews towards information gain), and UCB 1258 (e.g., Upper Confidence Bound, which includes an algorithm that balances exploration and exploitation by estimating the upper confidence bound of the expected reward for each action and selecting the action with the highest upper confidence bound).

FIG. 12D illustrates three graphs, where the top left graph is the percentage of top pairs discovered over 50 batches of experiments. Specifically, the top left graph indicates experimenters looking to a fraction of the pairs with the top 5 percentile of scores recovered by the algorithm capturing the ability to explore the high scoring regions. Further, the top right graph is the regret experienced by each method. Specifically, the top right graph shows an evaluation of the regret of each algorithm with respect to an optimal policy with access to the score matrix (e.g., the reward matrix shown below in FIG. 12E) to acquire the highest scoring pairs at each round. Lastly, the bottom middle graph is the performance of each method for known interactions. In other words, the bottom middle graph illustrates the number of known biological relations and how many each method is able to recover.

Experimenters found that the experimental implementation for the multi-perturbation interaction system 100 discovered pairs of genes that result in large norm interactions (e.g., biological activity beyond that expected from the individual knockouts themselves) significantly faster than random search, giving a 10% increase in the number of biological interactions that experimenters were able to discover after 50 rounds of experimentation. The relationships that experimenters detected were also complementary to that which would have been discovered using just single perturbations, and as a result, the two approaches can be combined to get a more detailed estimate of the relationships between genes from perturbation experiments.

As shown in FIG. 12D, the solid lines represent the mean performance, whereas the shaded region represent all the runs (min-max). As illustrated by FIG. 12D, the multi-perturbation interaction system 100 outperforms all the baselines significantly in terms of top scoring pairs discovered. Specifically, the top left graph illustrates that the multi-perturbation interaction system 100 discovers all the pairs with the top-5 percentile scores, whereas all the baselines barley recovers half of the top pairs.

Further, the multi-perturbation interaction system 100 outperforms other methods in terms of regret (e.g., cumulative difference between the performance of a model and the performance of the best possible action of the model in hindsight). In terms of regret, the multi-perturbation interaction system 100 outperforms the baselines, with the random method performing the worst. For instance, the top left graph demonstrates that the multi-perturbation interaction system 100 is able to exploit the low-rank structure in the reward matrix effectively.

In terms of known interactions, the multi-perturbation interaction system 100 still outperforms the baselines. As shown in the bottom middle graph for known interactions, the performance of all methods is quite similar, however the multi-perturbation interaction system 100 and the TS 1254 outperform all the baselines recovering around 10% more known relations.

As mentioned above, FIG. 12E illustrates three reward matrices: a first reward matrix 1270, generated utilizing a Matern 2.5 kernel to indicate measures of domain disjointedness, a second reward matrix 1272, generated utilizing an RBF kernel to indicate measures of domain disjointedness, and a third reward matrix 1274, generated to indicate measures of latent variable separability, in accordance with one or more embodiments. Specifically, for the set of 50 genes selected with a bias towards genes with known gene-gene interactions, FIG. 12E illustrates the final reward (e.g., the biological activity scores) for 1225 pairwise gene representations utilizing a variety of tests. FIG. 12E illustrates a scale on the right side of the graphs, indicated as R(i,j). Specifically, R(i,j) is the reward for each pair, where the darker the shade the lower reward (e.g., bottom of the scale) and the lighter the shade the higher the reward (e.g., top of the scale). Accordingly, FIG. 12E illustrates an example embodiment of the multi-perturbation interaction system 100 generating a matrix for perturbation pairs and utilizing active-matrix completion techniques to select an entry from the perturbation interaction matrix based on a predicted reward. Furthermore, as mentioned, the multi-perturbation interaction system 100 initiates downstream experimentation on a selected perturbation pair, determines a measure of biological activity for the selected pair, and updates the perturbation interaction matrix to include the actual measure of biological activity. In doing so, the multi-perturbation interaction system 100 iteratively fills in entries of the reward matrix by selecting an entry based on the predicted reward and information gain.

Additional information regarding an embodiment of the multi-perturbation interaction system 100 utilized to achieve the experimental results discussed above with regard to FIGS. 12A-12E will now be provided. Indeed, in some embodiments the multi-perturbation interaction system 100 computes statistics (e.g., a latent variable separability measure and a domain disjointedness measure) utilizing 1024-dimensional embeddings of original cell painting images extracted from a pre-trained autoencoder. Specifically, the multi-perturbation interaction system 100 derives the cell painting images from multi-cell images, with each single-cell nucleus centered within a 32×32 pixel box.

The multi-perturbation interaction system 100 can utilize an encoder of an NRE model to map single-cell images of shape (6, 32, 32) into a 128-dimensional feature vector. Further, the embodiment of the multi-perturbation interaction system 100 can utilize an encoder of the NRE model that consists of three convolutional blocks, wherein each layer includes a Conv2D layer with a 3×3 kernel, BatchNorm2D, ReLU activation, and MaxPool2D, and a progressively increasing number of channels from 6, to 32, to 64, to 128 while halving spatial dimensions at each of a plurality of max-pooling steps. After the convolutional layers, the multi-perturbation interaction system 100 flattens an output tensor of shape (128, 4, 4) to (2048) and passes the flattened output tensor through two fully connected blocks, wherein each of the two fully connected blocks includes a Linear layer, ReLU activation, and Dropout (with a dropout rate of 0.3). Accordingly, the multi-perturbation interaction system 100 transforms (e.g., reduces) a size of the flattened output tensor layer from 2048, to 256, to 128). Indeed, the embodiment of the multi-perturbation interaction system 100 can train the NRE model with an ADAM optimizer, utilizing a step size of 0.00005 for 5000 epochs and a batch size of 2048.

In some implementations, the multi-perturbation interaction system 100 determines the latent variable separability measure and the domain disjointedness measure as described by “Automated Discovery of Pairwise Interactions from Unstructured Data” found at https://arxiv.org/abs/2409.07594, authored by Zuheng Xu et. al, which is incorporated by reference herein in its entirety. Moreover, in some embodiments, the multi-perturbation interaction system 100 determines the latent variable separability measure as described in “Score-Based Interaction Testing in Pairwise Experiments,” found at https://openreview.net/forum?id=kalkk7yN3z&referrer=%5Bthe%20profile%20of%20Jason%20Hartford%5D(%2Fprofile%3Fid%3D-Jason_Hartford1), authored by Jana Osea et. al, which is incorporated by reference herein in its entirety.

Moreover, although the above discussion heavily involves gene-gene interactions, in one or more embodiments, the multi-perturbation interaction system 100 further utilizes the principles above for gene-drug interactions (or drug-drug interactions). Specifically, for a given gene knockout, the multi-perturbation interaction system 100 can compare the gene knockout to a library of available drugs. For instance, the multi-perturbation interaction system 100 (e.g., rather than running all pairs of genes and drugs) can generate measures of biological activity for existing gene-drug interactions and further generate a matrix that includes the existing data. Moreover, the multi-perturbation interaction system 100 can utilize the active-matrix completion techniques discussed above to select an entry of the perturbation interaction matrix that corresponds to a high potential gene-drug pair for further experimentation.

As alluded to above, the multi-perturbation interaction system 100 extends to exploration spaces that can contain nonlinear interactions. In other words, the multi-perturbation interaction system 100 can identify a variety of high potential pairs in a variety of different problems and more efficiently search the state space to surface predictions for further testing.

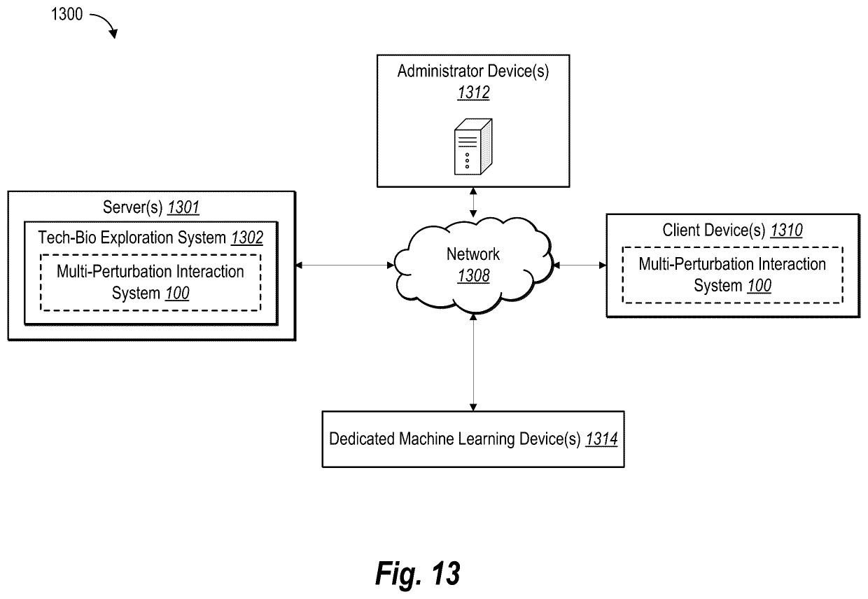

Additional detail regarding the multi-perturbation interaction system 100 environment will now be provided with reference to FIG. 13. In particular, FIG. 13 illustrates a schematic diagram of a system environment 1300 in which the multi-perturbation interaction system 100 can operate in accordance with one or more embodiments.

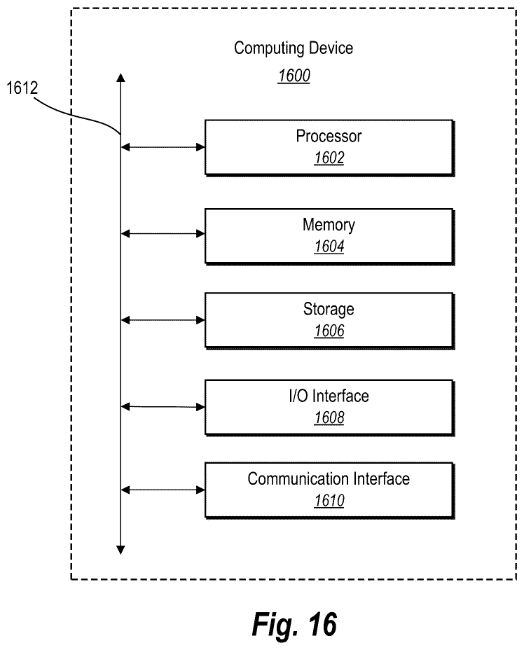

As shown in FIG. 13, the environment 1300 includes server(s) 1301 (which includes a tech-bio exploration system 1302 and the multi-perturbation interaction system 100), a network 1308, administrator device(s) 1312, dedicated machine learning device(s) 1314, and client device(s) 1310. As further illustrated in FIG. 13, the various computing devices within the environment can communicate via the network 1308. Although FIG. 13 illustrates the multi-perturbation interaction system 100 being implemented by a particular component and/or device within the environment, the multi-perturbation interaction system 100 can be implemented, in whole or in part, by other computing devices and/or components in the environment (e.g., the additional device(s)). Additional description regarding the illustrated computing devices is provided with respect to FIG. 16 below.

As shown in FIG. 13, the server(s) 1301 (e.g., one or more local servers operated by a particular entity) can include the tech-bio exploration system 1302. In some embodiments, the tech-bio exploration system 1302 can determine, store, generate, and/or display tech-bio information including maps of biology, experiments from various sources, and/or machine learning tech-bio predictions. For instance, the tech-bio exploration system 1302 can analyze data signals corresponding to various treatments or interventions (e.g., compounds or biologics) and the corresponding relationships in genetics, proteomics, phenomics (i.e., cellular phenotypes), and invivomics (e.g., expressions or results within a living animal). Moreover, the tech-bio exploration system 1302 provides an environment for operating, executing, and managing complex drug discovery pipelines.

For instance, the tech-bio exploration system 1302 can generate and access experimental results corresponding to gene sequences, protein shapes/folding, protein/compound interactions, phenotypes resulting from various interventions or perturbations (e.g., gene knockout sequences or compound treatments), and/or in vivo experimentation on various treatments in living animals. By analyzing these signals (e.g., utilizing various machine learning models), the tech-bio exploration system 1302 can generate or determine a variety of predictions and inter-relationships for improving treatments/interventions.

To illustrate, the tech-bio exploration system 1302 can generate maps of biology indicating biological inter-relationships or similarities between these various input signals to discover potential new treatments as part of the complex compound discovery process. For example, the tech-bio exploration system 1302 can utilize machine learning and/or maps of biology to identify a similarity between a first gene associated with disease treatment and a second gene previously unassociated with the disease based on a similarity in resulting phenotypes from gene knockout experiments. The tech-bio exploration system 1302 can then identify new treatments based on the gene similarity (e.g., by targeting compounds the impact the second gene). Similarly, the tech-bio exploration system 1302 can analyze signals from a variety of sources (e.g., protein interactions, or in vivo experiments) to predict efficacious treatments based on various levels of biological data.

The tech-bio exploration system 1302 can generate GUIs comprising dynamic user interface elements to convey tech-bio information and receive user input for intelligently exploring tech-bio information. Indeed, as mentioned above, the tech-bio exploration system 1302 can generate GUIs displaying different maps of biology that intuitively and efficiently express complex interactions between different biological systems for identifying improved treatment solutions. Furthermore, the tech-bio exploration system 1302 can also electronically communicate tech-bio information between various computing devices.

As shown in FIG. 13, the tech-bio exploration system 1302 can include a system that facilitates various models or algorithms for generating maps of biology (e.g., maps or visualizations illustrating similarities or relationships between genes, proteins, diseases, compounds, and/or treatments) and discovering new treatment options over one or more networks. For example, the tech-bio exploration system 1302 collects, manages, and transmits data across a variety of different entities, accounts, and devices. In some cases, the tech-bio exploration system 1302 is a network system that facilitates access to (and analysis of) tech-bio information within a centralized operating system. Indeed, the tech-bio exploration system 1302 can link data from different network-based research institutions to generate and analyze maps of biology.

As shown in FIG. 13, the tech-bio exploration system 1302 can include a system that comprises the multi-perturbation interaction system 100 that generates measures of biological activity between perturbation pairs (e.g., gene pair knockouts) and further selects one or more additional perturbation pairs (e.g., based on a balance between reward and exploration) to initiate additional downstream exploration. For example, in context of the above description for the tech-bio exploration system 1302, in some embodiments the tech-bio exploration system 1302 further utilizes the multi-perturbation interaction system 100 to enhance the coordination between various groups involved in the drug discovery process. For instance, the multi-perturbation interaction system 100 works in tandem with the tech-bio exploration system 1302 to identify perturbation pairs with a high potential with meaningful biological relationships (e.g., relationships between genes, proteins, diseases, compounds, and/or treatments). Further, the multi-perturbation interaction system 100 can utilize the identified perturbation pairs with high potential to initiate additional experimentation to efficiently explore a perturbation pair space for developing biological compounds (e.g., to target one or more perturbation pairs). Specifically, the tech-bio exploration system 1302 utilizes the multi-perturbation interaction system 100 to generate measures of biological activity, map the measures of biological activity to a matrix and further utilize an active-matrix completion algorithm to make one or more selections for exploring a perturbation pair. Additionally, in some embodiments, the multi-perturbation interaction system 100 generates additional measures of biological activity based on the additional exploration, updates the perturbation interaction matrix, and further selects another entry from the perturbation interaction matrix.

To further illustrate, the tech-bio exploration system 1302 utilizes the multi-perturbation interaction system 100 at the program discovery phase to identify compounds that target certain genes. For instance, the multi-perturbation interaction system 100 can test various hypotheses for how a double perturbation (e.g., a double gene knockout) affects a cell (e.g., via synthetic lethality or morphological/phenomic changes) and further utilizes the multi-perturbation interaction system 100 to efficiently explore the state space without performing brute-force searches.

As also illustrated in FIG. 13, the environment includes the client device(s) 1310. As mentioned above, the client device(s) 1310 can be involved in the process of drug discovery. Thus, for example, the client device(s) 1310 can coordinate/manage generating measures of biological activity, feeding the measures to an additional client device and further initiating the selection of an additional perturbation pair for downstream experimentation. For instance, the client device(s) 1310 can coordinate/manage testing perturbation pairs under various conditions to further determine whether to initiate one or more programs (industrial program generation or industrial compound generation) for one or more of the perturbation pairs (e.g., developing drug compounds that target the perturbation pair).

To illustrate, the client device(s) 1310 can include computing devices that implement or manage a compound program generation stage of a compound discovery process. Similarly, the client device(s) 1310 can include computing devices that implement or manage a compound lead generation stage and the client device(s) 1310 can include computing devices that implement or manage a compound/dose selection stage. For example, the multi-perturbation interaction system 100 can receive one or more requests to make one or more selections of perturbation pairs based on the existing data related to measures of biological activities.

In some embodiments, the environment also includes additional device(s). For example, the multi-perturbation interaction system 100 can utilize the additional device(s) to further operate and manage downstream operations after generating measures of biological activity and selecting an additional perturbation pair. For instance, the additional device(s) include the experimental device(s) 1310 (e.g., to expose a cell to the additional perturbation pair) and analytical device(s) (e.g., to analyze the exposed cell, generate an image of the exposed cell, etc.). Further, in some instances, the additional device(s) also include the computing devices discussed below in FIG. 16.

Furthermore, in one or more implementations, the client device(s) 1310 include a client application. The client application can include instructions that (upon execution) cause the client device(s) 1310 to perform various actions. For example, a user of a user account can interact with the client application on the client device(s) 1310 to execute the generation of a matrix that includes a plurality of measures of biological activities and to begin an active-matrix completion task. For instance, in some embodiments the multi-perturbation interaction system 100 receives a request to generate a measure of biological activity from experimental data that includes sets of individual representations and a pairwise representation. In response, the multi-perturbation interaction system 100 can generate the measure of biological activity and provide an option for the client device(s) 1310 to identify high potential perturbation pairs based on the existing data. In some instances, the multi-perturbation interaction system 100 selects a perturbation pair and in response, further causes the client device(s) 1310 to further present options for executing an action (e.g., performing downstream experiments, tests, or evaluations for the selected perturbation pair).

Although not shown, the environment can also include dedicated training device(s). For example, the dedicated training device(s) can include computing devices or virtual machines dedicated to training or implementing a generative stochastic model (e.g., for exploring a state space), a machine learning model (e.g., for generating embeddings), a pairwise prediction model, and an active-matrix completion algorithm. For example, the dedicated training device(s) can provide datasets, parameters, objectives, and other learning constraints to train and/or implement the aforementioned models. Thus, the multi-perturbation interaction system 100 interacts with the dedicated training device(s) to learn certain state spaces and to accurately generate corresponding outputs.

The environment can also include experimental device(s) 1310. For example, the tech-bio exploration system 1302 can interact with the experimental device(s) 1310 that include intelligent robotic devices and camera devices for generating and capturing digital images of cellular phenotypes resulting from different perturbations (e.g., genetic knockouts or compound treatments of stem cells). Similarly, the experimental device(s) 1310 can include camera devices and/or other sensors (e.g., heat or motion sensors) capturing real-time information from animals as part of in vivo experimentation. The tech-bio exploration system 1302 can also interact with a variety of other experimental device(s) 1310 such as devices for determining, generating, or extracting gene sequences or protein information. For example, the experimental device(s) 1310 may include computing devices linked to biosensorselectrophysiological platforms, x-ray crystallography machines, liquid chromatography mass spectrometry systems, nuclear magnetic resonance spectrometers, mass spectrometers. In some implementations, the multi-perturbation interaction system 100 selects a perturbation pair and further determines to employ or utilize one or more experimental devices (e.g., to initiate one or more experiments based on the selection).

To illustrate, the client device(s) 1310 can include computing devices that implement or manage a compound program generation stage of a compound discovery process. Similarly, the client device(s) 1310 can include computing devices that implement or manage a compound lead generation stage and the client device(s) 1310 can include computing devices that implement or manage a compound/dose selection stage. For example, the multi-perturbation interaction system 100 can receive one or more requests to utilize the dedicated machine learning device(s) 1314 to generate a pairwise representation. For instance, the multi-perturbation interaction system 100 can receive additional requests from the client device(s) 1310 that include a measure of biological activity for the pairwise representation.

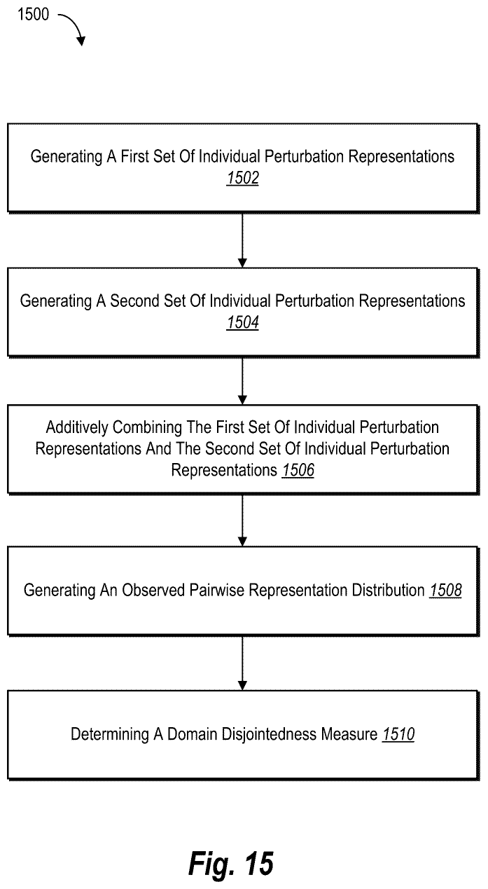

As further shown in FIG. 13, the environment includes the network 1308. As mentioned above, the network 1308 can enable communication between components of the environment. In one or more embodiments, the network 1308 may include a suitable network and may communicate using a various number of communication platforms and technologies suitable for transmitting data and/or communication signals, examples of which are described with reference to FIG. 15. Furthermore, although FIG. 13 illustrates computing devices communicating via the network 1308, the various components of the environment can communicate and/or interact via other methods (e.g., communicate directly).

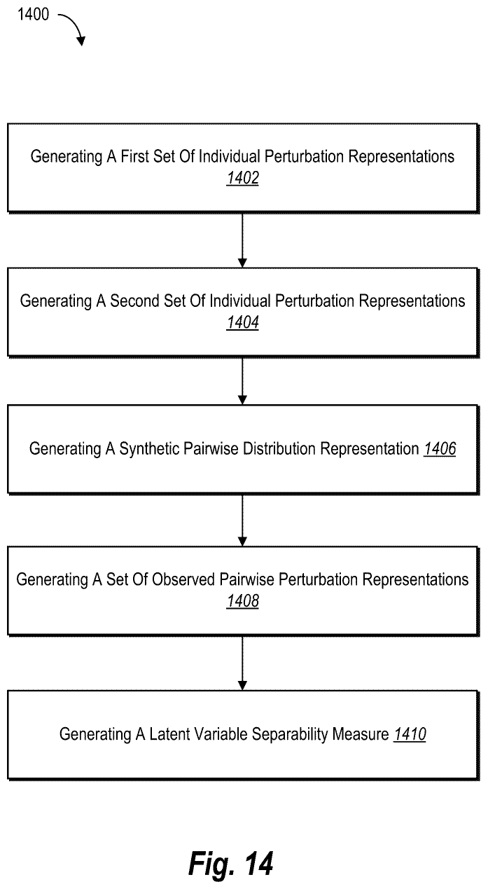

FIGS. 1-13, the corresponding text, and the examples provide a number of different systems, methods, and non-transitory computer readable media for generating a measure of biological activity from comparing individual perturbation representations and a pairwise representation. In addition to the foregoing, embodiments can also be described in terms of flowcharts comprising acts for accomplishing a particular result. For example, FIGS. 14-15 illustrates flowcharts of an example sequence of acts in accordance with one or more embodiments.

While FIGS. 14-15 illustrate acts according to some embodiments, alternative embodiments may omit, add to, reorder, and/or modify any of the acts shown in FIGS. 14-15. The acts of FIGS. 14-15 can be performed as part of a method (e.g., a computer-implemented method). Alternatively, a non-transitory computer readable medium can comprise instructions, that when executed by one or more processors (e.g., at least one processor), cause a computing device to perform the acts of FIGS. 14-15. In still further embodiments, a system can perform the acts of FIGS. 14-15. Additionally, the acts described herein may be repeated or performed in parallel with one another or in parallel with different instances of the same or other similar acts.

FIG. 14 illustrates an example series of acts 1400 for generating a latent variable separability measure in accordance with one or more embodiments. The series of acts 1400 can include an act 1402 of generating a first set of individual perturbation representations, an act 1404 of generating a second set of individual perturbation representations, an act 1406 of generating a synthetic pairwise distribution representation, an act 1408 of generating a set of observed pairwise perturbation representations, and an act 1410 of generating a latent variable separability measure.

Specifically, the series of acts 1400 can include acts 1402-1410 of generating a first set of individual perturbation representations of a first set of cells exposed to a first perturbation; generating a second set of individual perturbation representations of a second set of cells exposed to a second perturbation; generating a synthetic pairwise distribution representation from the first set of individual perturbation representations and the second set of individual perturbation representations; generating a set of observed pairwise perturbation representations of a third set of cells exposed to both the first perturbation and the second perturbation; and generating a latent variable separability measure between the first perturbation and the second perturbation by comparing the synthetic pairwise distribution representation and the set of observed pairwise perturbation representations.

For example, in one or more embodiments, the series of acts 1400 includes generating a first individual perturbation distribution density ratio metric from the first set of individual perturbation representations, wherein the first individual perturbation distribution density ratio metric indicates a density ratio between a first distribution corresponding to the first set of individual perturbation representations and a control distribution 308 corresponding to a control set of representations of a control set of cells.

In addition, in one or more embodiments, the series of acts 1400 includes generating the first set of individual perturbation representations by generating, utilizing an encoder, a first set of feature vectors of a first set of digital images portraying the first set of cells. Further, in some embodiments, the series of acts 1400 includes generating a log-density ratio from the first set of feature vectors utilizing a ratio density machine learning model. Moreover, in one or more embodiments, the series of acts 1400 includes generating the first individual perturbation distribution density ratio metric by generating a KL-divergence metric for the first set of individual perturbation representations from the log-density ratio.

Additionally, in some embodiments, the series of acts 1400 includes generating a second individual perturbation distribution density ratio metric from the second set of individual perturbation representations. Moreover, in one or more embodiments, the series of acts 1400 includes combining the first individual perturbation distribution density ratio metric and the second individual perturbation distribution density ratio metric to generate the synthetic pairwise distribution representation.

Further, in one or more embodiments, the series of acts 1400 includes generating the set of observed pairwise perturbation representations by utilizing an encoder to generate a third set of feature vectors from a set of images portraying the third set of cells exposed to both the first perturbation and the second perturbation. Additionally, in some embodiments, the series of acts 1400 includes generating, a third individual distribution density ratio metric from the third set of feature vectors. In addition, in one or more embodiments, the series of acts 1400 includes generating the latent variable separability measure by comparing the third individual distribution density ratio metric and the synthetic pairwise distribution representation.

Moreover, in some embodiments, the series of acts 1400 includes generating a first score function indicating a first gradient of a first probability density of the first set of individual perturbation representations. Additionally, in one or more embodiments, the series of acts 1400 includes generating a second score function indicating a second gradient of a second probability density of the second set of individual perturbation representations. Further, in some embodiments, the series of acts 1400 includes generating the synthetic pairwise distribution representation comprises generating a combined score function from the first score function and the second score function.

In addition, in one or more embodiments, the series of acts 1400 includes generating a pairwise score function from the set of observed pairwise perturbation representations of the third set of cells exposed to the first perturbation and the second perturbation. Indeed, in some embodiments, the series of acts 1400 includes comparing the combined score function and the pairwise score function to generate the latent variable separability measure.

Additionally, in some embodiments, the series of acts 1400 includes generating a Kernelized Stein Discrepancy metric by comparing the set of observed pairwise perturbation representations and the combined score function.

Further, in one or more embodiments, the series of acts 1400 includes generating a perturbation interaction matrix from latent variable separability measures corresponding to a plurality of perturbation pairs, wherein latent variable separability measures comprise the latent variable separability measure corresponding to the first perturbation and the second perturbation. Moreover, in some embodiments, the series of acts 1400 includes utilizing an active matrix completion algorithm to select a perturbation pair for additional experimentation based on the perturbation interaction matrix

FIG. 15 illustrates an example series of acts 1500 for determining a domain disjointedness measure in accordance with one or more embodiments. The series of acts 1500 can include an act 1502 of generating a first set of individual perturbation representations; an act 1504 of generating a second set of individual perturbation representations, an act 1506 of additively combining the first set of individual perturbation representations and the second set of individual perturbation representations; an act 1508 of generating an observed pairwise representation distribution, and an act 1510 of determining a domain disjointedness measure. Specifically, the series of acts 1500 can include acts 1502-1510 of generating a first set of individual perturbation representations of a first set of cells exposed to a first perturbation; generating a second set of individual perturbation representations of a second set of cells exposed to a second perturbation; additively combining the first set of individual perturbation representations and the second set of individual perturbation representations to generate a synthetic pairwise perturbation distribution; generating an observed pairwise representation distribution from a third set of representations of a third set of cells exposed to both the first perturbation and the second perturbation; and determining a domain disjointedness measure between the first perturbation and the second perturbation by comparing the observed pairwise representation distribution and the synthetic pairwise perturbation distribution.

For example, in one or more embodiments, the series of acts 1500 includes determining the domain disjointedness measure between the first perturbation and the second perturbation by determining a degree to which the first perturbation and the second perturbation compose additively to form the synthetic pairwise perturbation distribution.

Further, in some embodiments, the series of acts 1500 includes generating the first set of individual perturbation representations by generating, utilizing an encoder, a first set of feature vectors from digital images portraying the first set of cells exposed to the first perturbation. Additionally, in one or more embodiments, the series of acts 1500 includes generating the second set of individual perturbation representations by generating, utilizing the encoder, a second set of feature vectors from digital images portraying the second set of cells exposed to the first perturbation.

Moreover, in some embodiments, the series of acts 1500 includes generating the synthetic pairwise perturbation distribution by additively combining pairwise samples from the first set of feature vectors and the second set of feature vectors.

In addition, in one or more embodiments, the series of acts 1500 can include mapping the synthetic pairwise perturbation distribution to a reproducing kernel space. Indeed, in some embodiments, the series of acts 1500 can include mapping the observed pairwise representation distribution to the reproducing kernel space. Additionally, in one or more embodiments, the series of acts 1500 can include comparing the synthetic pairwise perturbation distribution and the observed pairwise representation distribution in the reproducing kernel space.

Further, in some embodiments, the series of acts 1500 can include determining a mean discrepancy metric between the observed pairwise representation distribution and the synthetic pairwise perturbation distribution within a reproducing kernel space.

Moreover, in one or more embodiments, the series of acts 1500 can include generating a perturbation interaction matrix from a plurality of domain disjointedness measures corresponding to a plurality of perturbation pairs, wherein the plurality of domain disjointedness measures comprise the domain disjointedness measure corresponding to the first perturbation and the second perturbation. In addition, the series of acts 1500 can include generating, utilizing a pairwise prediction model, a plurality of predicted pairwise perturbation interaction scores and corresponding information gain predictions utilizing the perturbation interaction matrix.

Additionally, in one or more embodiments, the series of acts 1500 includes utilizing an active-matrix completion algorithm to select a perturbation pair based on the plurality of predicted pairwise perturbation interaction scores and the corresponding information gain predictions.

Further, in some embodiments, the series of acts 1500 includes transmitting the perturbation pair to initiate additional experimental processes for generating an additional domain disjointedness measure for the perturbation pair. Additionally, in one or more embodiments, the series of acts 1500 includes updating the perturbation interaction matrix based on the additional domain disjointedness measure for the perturbation pair.

Embodiments of the present disclosure may comprise or utilize a special purpose or general-purpose computer including computer hardware, such as, for example, one or more processors and system memory, as discussed in greater detail below. Embodiments within the scope of the present disclosure also include physical and other computer-readable media for carrying or storing computer-executable instructions and/or data structures. In particular, one or more of the processes described herein may be implemented at least in part as instructions embodied in a non-transitory computer-readable medium and executable by one or more computing devices (e.g., any of the media content access devices described herein). In general, a processor (e.g., a microprocessor) receives instructions, from a non-transitory computer-readable medium, (e.g., memory), and executes those instructions, thereby performing one or more processes, including one or more of the processes described herein.

Computer-readable media can be any available media that can be accessed by a general purpose or special purpose computer system. Computer-readable media that store computer-executable instructions are non-transitory computer-readable storage media (devices). Computer-readable media that carry computer-executable instructions are transmission media. Thus, by way of example, and not limitation, embodiments of the disclosure can comprise at least two distinctly different kinds of computer-readable media: non-transitory computer-readable storage media (devices) and transmission media.

Non-transitory computer-readable storage media (devices) includes RAM, ROM, EEPROM, CD-ROM, solid state drives (“SSDs”) (e.g., based on RAM), Flash memory, phase-change memory (“PCM”), other types of memory, other optical disk storage, magnetic disk storage or other magnetic storage devices, or any other medium which can be used to store desired program code means in the form of computer-executable instructions or data structures and which can be accessed by a general purpose or special purpose computer.

A “network” is defined as one or more data links that enable the transport of electronic data between computer systems and/or modules and/or other electronic devices. When information is transferred or provided over a network or another communications connection (either hardwired, wireless, or a combination of hardwired or wireless) to a computer, the computer properly views the connection as a transmission medium. Transmissions media can include a network and/or data links which can be used to carry desired program code means in the form of computer-executable instructions or data structures and which can be accessed by a general purpose or special purpose computer. Combinations of the above should also be included within the scope of computer-readable media.

Further, upon reaching various computer system components, program code means in the form of computer-executable instructions or data structures can be transferred automatically from transmission media to non-transitory computer-readable storage media (devices) (or vice versa). For example, computer-executable instructions or data structures received over a network or data link can be buffered in RAM within a network interface module (e.g., a “NIC”), and then eventually transferred to computer system RAM and/or to less volatile computer storage media (devices) at a computer system. Thus, it should be understood that non-transitory computer-readable storage media (devices) can be included in computer system components that also (or even primarily) utilize transmission media.

Computer-executable instructions comprise, for example, instructions and data which, when executed by a processor, cause a general-purpose computer, special purpose computer, or special purpose processing device to perform a certain function or group of functions. In some embodiments, computer-executable instructions are executed by a general-purpose computer to turn the general-purpose computer into a special purpose computer implementing elements of the disclosure. The computer-executable instructions may be, for example, binaries, intermediate format instructions such as assembly language, or even source code. Although the subject matter has been described in language specific to structural features and/or methodological acts, it is to be understood that the subject matter defined in the appended claims is not necessarily limited to the described features or acts described above. Rather, the described features and acts are disclosed as example forms of implementing the claims.

Those skilled in the art will appreciate that the disclosure may be practiced in network computing environments with many types of computer system configurations, including, personal computers, desktop computers, laptop computers, message processors, hand-held devices, multi-processor systems, microprocessor-based or programmable consumer electronics, network PCs, minicomputers, mainframe computers, mobile telephones, PDAs, tablets, pagers, routers, switches, and the like. The disclosure may also be practiced in distributed system environments where local and remote computer systems, which are linked (either by hardwired data links, wireless data links, or by a combination of hardwired and wireless data links) through a network, both perform tasks. In a distributed system environment, program modules may be located in both local and remote memory storage devices.

Embodiments of the present disclosure can also be implemented in cloud computing environments. As used herein, the term “cloud computing” refers to a model for enabling on-demand network access to a shared pool of configurable computing resources. For example, cloud computing can be employed in the marketplace to offer ubiquitous and convenient on-demand access to the shared pool of configurable computing resources. The shared pool of configurable computing resources can be rapidly provisioned via virtualization and released with low management effort or service provider interaction, and then scaled accordingly.

A cloud-computing model can be composed of various characteristics such as, for example, on-demand self-service, broad network access, resource pooling, rapid elasticity, measured service, and so forth. A cloud-computing model can also expose various service models, such as, for example, Software as a Service (“SaaS”), Platform as a Service (“PaaS”), and Infrastructure as a Service (“IaaS”). A cloud-computing model can also be deployed using different deployment models such as private cloud, community cloud, public cloud, hybrid cloud, and so forth. In addition, as used herein, the term “cloud-computing environment” refers to an environment in which cloud computing is employed.

FIG. 15 illustrates a block diagram of an example computing device 1600 that may be configured to perform one or more of the processes described above. One will appreciate that one or more computing devices, such as the computing device 1600 may represent the computing devices described above. In one or more embodiments, the computing device 1600 may be a mobile device (e.g., a mobile telephone, a smartphone, a PDA, a tablet, a laptop, a camera, a tracker, a watch, a wearable device, etc.). In some embodiments, the computing device 1600 may be a non-mobile device (e.g., a desktop computer or another type of client device). Further, the computing device 1600 may be a server device that includes cloud-based processing and storage capabilities.

As shown in FIG. 15, the computing device 1600 can include one or more processor(s) 1602, memory 1604, a storage device 1606, input/output interfaces 1608 (or “I/O interfaces 1608”), and a communication interface 1610, which may be communicatively coupled by way of a communication infrastructure (e.g., bus 1612). While the computing device 1600 is shown in FIG. 15, the components illustrated in FIG. 15 are not intended to be limiting. Additional or alternative components may be used in other embodiments. Furthermore, in certain embodiments, the computing device 1600 includes fewer components than those shown in FIG. 15. Components of the computing device 1600 shown in FIG. 15 will now be described in additional detail.

In particular embodiments, the processor(s) 1602 includes hardware for executing instructions, such as those making up a computer program. As an example, and not by way of limitation, to execute instructions, the processor(s) 1602 may retrieve (or fetch) the instructions from an internal register, an internal cache, memory 1604, or a storage device 1606 and decode and execute them.

The computing device 1600 includes memory 1604, which is coupled to the processor(s) 1602. The memory 1604 may be used for storing data, metadata, and programs for execution by the processor(s). The memory 1604 may include one or more of volatile and non-volatile memories, such as Random-Access Memory (“RAM”), Read-Only Memory (“ROM”), a solid-state disk (“SSD”), Flash, Phase Change Memory (“PCM”), or other types of data storage. The memory 1604 may be internal or distributed memory.

The computing device 1600 includes a storage device 1606 includes storage for storing data or instructions. As an example, and not by way of limitation, the storage device 1606 can include a non-transitory storage medium described above. The storage device 1606 may include a hard disk drive (HDD), flash memory, a Universal Serial Bus (USB) drive or a combination these or other storage devices.

As shown, the computing device 1600 includes one or more I/O interfaces 1608, which are provided to allow a user to provide input to (such as user strokes), receive output from, and otherwise transfer data to and from the computing device 1600. These I/O interfaces 1608 may include a mouse, keypad or a keyboard, a touch screen, camera, optical scanner, network interface, modem, other known I/O devices or a combination of such I/O interfaces 1608. The touch screen may be activated with a stylus or a finger.

The I/O interfaces 1608 may include one or more devices for presenting output to a user, including, but not limited to, a graphics engine, a display (e.g., a display screen), one or more output drivers (e.g., display drivers), one or more audio speakers, and one or more audio drivers. In certain embodiments, I/O interfaces 1608 are configured to provide graphical data to a display for presentation to a user. The graphical data may be representative of one or more graphical user interfaces and/or any other graphical content as may serve a particular implementation.

The computing device 1600 can further include a communication interface 1610. The communication interface 1610 can include hardware, software, or both. The communication interface 1610 provides one or more interfaces for communication (such as, for example, packet-based communication) between the computing device and one or more other computing devices or one or more networks. As an example, and not by way of limitation, communication interface 1610 may include a network interface controller (NIC) or network adapter for communicating with an Ethernet or other wire-based network or a wireless NIC (WNIC) or wireless adapter for communicating with a wireless network, such as a WI-FI. The computing device 1600 can further include a bus 1612. The bus 1612 can include hardware, software, or both that connects components of computing device 1600 to each other.

In one or more implementations, various computing devices can communicate over a computer network. This disclosure contemplates any suitable network. As an example, and not by way of limitation, one or more portions of a network may include an ad hoc network, an intranet, an extranet, a virtual private network (“VPN”), a local area network (“LAN”), a wireless LAN (“WLAN”), a wide area network (“WAN”), a wireless WAN (“WWAN”), a metropolitan area network (“MAN”), a portion of the Internet, a portion of the Public Switched Telephone Network (“PSTN”), a cellular telephone network, or a combination of two or more of these.

In particular embodiments, the computing device 1600 can include a client device that includes a requester application or a web browser, such as MICROSOFT INTERNET EXPLORER, GOOGLE CHROME, or MOZILLA FIREFOX, and may have one or more add-ons, plug-ins, or other extensions, such as TOOLBAR or YAHOO TOOLBAR. A user at the client device may enter a Uniform Resource Locator (“URL”) or other address directing the web browser to a particular server (such as server), and the web browser may generate a Hyper Text Transfer Protocol (“HTTP”) request and communicate the HTTP request to server. The server may accept the HTTP request and communicate to the client device one or more Hyper Text Markup Language (“HTML”) files responsive to the HTTP request. The client device may render a webpage based on the HTML files from the server for presentation to the user. This disclosure contemplates any suitable webpage files. As an example, and not by way of limitation, webpages may render from HTML files, Extensible Hyper Text Markup Language (“XHTML”) files, or Extensible Markup Language (“XML”) files, according to particular needs. Such pages may also execute scripts such as, for example and without limitation, those written in JAVASCRIPT, JAVA, MICROSOFT SILVERLIGHT, combinations of markup language and scripts such as AJAX (Asynchronous JAVASCRIPT and XML), and the like. Herein, reference to a webpage encompasses one or more corresponding webpage files (which a browser may use to render the webpage) and vice versa, where appropriate.

In particular embodiments, the tech-bio exploration system 1302 may include a variety of servers, sub-systems, programs, modules, logs, and data stores. In particular embodiments, the tech-bio exploration system 1302 may include one or more of the following: a web server, action logger, API-request server, transaction engine, cross-institution network interface manager, notification controller, action log, third-party-content-object-exposure log, inference module, authorization/privacy server, search module, user-interface module, user-profile (e.g., provider profile or requester profile) store, connection store, third-party content store, or location store. The tech-bio exploration system 1302 may also include suitable components such as network interfaces, security mechanisms, load balancers, failover servers, management-and-network-operations consoles, other suitable components, or any suitable combination thereof. In particular embodiments, the tech-bio exploration system 1302 may include one or more user-profile stores for storing user profiles and/or account information for credit accounts, secured accounts, secondary accounts, and other affiliated financial networking system accounts. A user profile may include, for example, biographic information, demographic information, financial information, behavioral information, social information, or other types of descriptive information, such as interests, affinities, or location.

The web server may include a mail server or other messaging functionality for receiving and routing messages between the tech-bio exploration system 1302 and one or more client devices. An action logger may be used to receive communications from a web server about a user's actions on or off the tech-bio exploration system 1302. In conjunction with the action log, a third-party-content-object log may be maintained of user exposures to third-party-content objects. A notification controller may provide information regarding content objects to a client device. Information may be pushed to a client device as notifications, or information may be pulled from a client device responsive to a request received from the client device. Authorization servers may be used to enforce one or more privacy settings of the users of the tech-bio exploration system 1302. A privacy setting of a user determines how particular information associated with a user can be shared. The authorization server may allow users to opt in to or opt out of having their actions logged by the tech-bio exploration system 1302 or shared with other systems, such as, for example, by setting appropriate privacy settings. Third-party-content-object stores may be used to store content objects received from third parties. Location stores may be used for storing location information received from a client device associated with users.

In the foregoing specification, the invention has been described with reference to specific example embodiments thereof. Various embodiments and aspects of the invention(s) are described with reference to details discussed herein, and the accompanying drawings illustrate the various embodiments. The description above and drawings are illustrative of the invention and are not to be construed as limiting the invention. Numerous specific details are described to provide a thorough understanding of various embodiments of the present invention.

The present invention may be embodied in other specific forms without departing from its spirit or essential characteristics. The described embodiments are to be considered in all respects only as illustrative and not restrictive. For example, the methods described herein may be performed with less or more steps/acts or the steps/acts may be performed in differing orders. Additionally, the steps/acts described herein may be repeated or performed in parallel to one another or in parallel to different instances of the same or similar steps/acts. The scope of the invention is, therefore, indicated by the appended claims rather than by the foregoing description. All changes that come within the meaning and range of equivalency of the claims are to be embraced within their scope.

What is claimed is:

1. A computer-implemented method comprising:
generating a first set of individual perturbation representations of a first set of cells exposed to a first perturbation;
generating a second set of individual perturbation representations of a second set of cells exposed to a second perturbation;
generating a synthetic pairwise distribution representation from the first set of individual perturbation representations and the second set of individual perturbation representations;
generating a set of observed pairwise perturbation representations of a third set of cells exposed to both the first perturbation and the second perturbation; and
generating a latent variable separability measure between the first perturbation and the second perturbation by comparing the synthetic pairwise distribution representation and the set of observed pairwise perturbation representations.

2. The computer-implemented method of claim 1, wherein generating the synthetic pairwise distribution representation comprises: generating a first individual perturbation distribution density ratio metric from the first set of individual perturbation representations, wherein the first individual perturbation distribution density ratio metric indicates a density ratio between a first distribution corresponding to the first set of individual perturbation representations and a control distribution corresponding to a control set of representations of a control set of cells.

3. The computer-implemented method of claim 2, further comprising generating the first individual perturbation distribution density ratio metric by:
generating the first set of individual perturbation representations by generating, utilizing an encoder, a first set of feature vectors of a first set of digital images portraying the first set of cells;
generating a log-density ratio from the first set of feature vectors utilizing a ratio density machine learning model; and
generating the first individual perturbation distribution density ratio metric by generating a KL-divergence metric for the first set of individual perturbation representations from the log-density ratio.

4. The computer-implemented method of claim 2, wherein generating the synthetic pairwise distribution representation comprises:
generating a second individual perturbation distribution density ratio metric from the second set of individual perturbation representations; and
combining the first individual perturbation distribution density ratio metric and the second individual perturbation distribution density ratio metric to generate the synthetic pairwise distribution representation.

5. The computer-implemented method of claim 2, further comprising:
generating the set of observed pairwise perturbation representations by utilizing an encoder to generate a third set of feature vectors from a set of images portraying the third set of cells exposed to both the first perturbation and the second perturbation;
generating, a third individual distribution density ratio metric from the third set of feature vectors; and
generating the latent variable separability measure by comparing the third individual distribution density ratio metric and the synthetic pairwise distribution representation.

6. The computer-implemented method of claim 1, further comprising:
generating a first score function indicating a first gradient of a first probability density of the first set of individual perturbation representations;
generating a second score function indicating a second gradient of a second probability density of the second set of individual perturbation representations; and
generating the synthetic pairwise distribution representation comprises generating a combined score function from the first score function and the second score function.

7. The computer-implemented method of claim 6, further comprising:
generating a pairwise score function from the set of observed pairwise perturbation representations of the third set of cells exposed to the first perturbation and the second perturbation; and
comparing the combined score function and the pairwise score function to generate the latent variable separability measure.

8. The computer-implemented method of claim 6, wherein generating the latent variable separability measure comprises generating a Kernelized Stein Discrepancy metric by comparing the set of observed pairwise perturbation representations and the combined score function.

9. The computer-implemented method of claim 1, further comprising:
generating a perturbation interaction matrix from latent variable separability measures corresponding to a plurality of perturbation pairs, wherein latent variable separability measures comprise the latent variable separability measure corresponding to the first perturbation and the second perturbation; and
utilizing an active matrix completion algorithm to select a perturbation pair for additional experimentation based on the perturbation interaction matrix.

10. A system comprising;
at least one processor; and
at least one non-transitory computer-readable storage medium storing instructions that, when executed by the at least one processor, cause the system to:
generate a first set of individual perturbation representations of a first set of cells exposed to a first perturbation;
generate a second set of individual perturbation representations of a second set of cells exposed to a second perturbation;
generate a synthetic pairwise distribution representation from the first set of individual perturbation representations and the second set of individual perturbation representations;
generate a set of observed pairwise perturbation representations of a third set of cells exposed to both the first perturbation and the second perturbation; and
generate a latent variable separability measure between the first perturbation and the second perturbation by comparing the synthetic pairwise distribution representation and the set of observed pairwise perturbation representations.

11. The system of claim 10, further comprising instructions that, when executed by the at least one processor, cause the system to generate the synthetic pairwise distribution representation by: generating a first individual perturbation distribution density ratio metric from the first set of individual perturbation representations, wherein the first individual perturbation distribution density ratio metric indicates a density ratio between a first distribution corresponding to the first set of individual perturbation representations and a control distribution corresponding to a control set of representations of a control set of cells.

12. The system of claim 11, further comprising instructions that, when executed by the at least one processor, cause the system to generate the first individual perturbation distribution density ratio metric by:
generating the first set of individual perturbation representations by generating, utilizing an encoder, a first set of feature vectors of a first set of digital images portraying the first set of cells;
generating a log-density ratio from the first set of feature vectors utilizing a ratio density machine learning model; and
generating the first individual perturbation distribution density ratio metric by generating a KL-divergence metric for the first set of individual perturbation representations from the log-density ratio.

13. The system of claim 11, further comprising instructions that, when executed by the at least one processor, cause the system to generate the synthetic pairwise distribution representation by:
generating a second individual perturbation distribution density ratio metric from the second set of individual perturbation representations; and
combining the first individual perturbation distribution density ratio metric and the second individual perturbation distribution density ratio metric to generate the synthetic pairwise distribution representation.

14. The system of claim 11, further comprising instructions that, when executed by the at least one processor, cause the system to:
generate the set of observed pairwise perturbation representations by utilizing an encoder to generate a third set of feature vectors from a set of images portraying the third set of cells exposed to both the first perturbation and the second perturbation;
generate, a third individual distribution density ratio metric from the third set of feature vectors; and
generate the latent variable separability measure by comparing the third individual distribution density ratio metric and the synthetic pairwise distribution representation.

15. The system of claim 10, further comprising instructions that, when executed by the at least one processor, cause the system to:
generate a first score function indicating a first gradient of a first probability density of the first set of individual perturbation representations;
generate a second score function indicating a second gradient of a second probability density of the second set of individual perturbation representations; and
generate the synthetic pairwise distribution representation by generating a combined score function from the first score function and the second score function.

16. A non-transitory computer-readable medium storing instructions that, when executed by at least one processor, cause a computing device to:
generate a first set of individual perturbation representations of a first set of cells exposed to a first perturbation;
generate a second set of individual perturbation representations of a second set of cells exposed to a second perturbation;
generate a synthetic pairwise distribution representation from the first set of individual perturbation representations and the second set of individual perturbation representations;
generate a set of observed pairwise perturbation representations of a third set of cells exposed to both the first perturbation and the second perturbation; and
generate a latent variable separability measure between the first perturbation and the second perturbation by comparing the synthetic pairwise distribution representation and the set of observed pairwise perturbation representations.

17. The non-transitory computer-readable medium of claim 16, further comprising instructions that, when executed by the at least one processor, cause the computing device to generate the synthetic pairwise distribution representation by: generating a first individual perturbation distribution density ratio metric from the first set of individual perturbation representations, wherein the first individual perturbation distribution density ratio metric indicates a density ratio between a first distribution corresponding to the first set of individual perturbation representations and a control distribution corresponding to a control set of representations of a control set of cells.

18. The non-transitory computer-readable medium of claim 17, further comprising instructions that, when executed by the at least one processor, cause the computing device to generate the first individual perturbation distribution density ratio metric by:
generating the first set of individual perturbation representations by generating, utilizing an encoder, a first set of feature vectors of a first set of digital images portraying the first set of cells;
generating a log-density ratio from the first set of feature vectors utilizing a ratio density machine learning model; and
generating the first individual perturbation distribution density ratio metric by generating a KL-divergence metric for the first set of individual perturbation representations from the log-density ratio.

19. The non-transitory computer-readable medium of claim 17, further comprising instructions that, when executed by the at least one processor, cause the computing device to generate the synthetic pairwise distribution representation by:
generating a second individual perturbation distribution density ratio metric from the second set of individual perturbation representations; and
combining the first individual perturbation distribution density ratio metric and the second individual perturbation distribution density ratio metric to generate the synthetic pairwise distribution representation.

20. The non-transitory computer-readable medium of claim 17, further comprising instructions that, when executed by the at least one processor, cause the computing device to:
generate the set of observed pairwise perturbation representations by utilizing an encoder to generate a third set of feature vectors from a set of images portraying the third set of cells exposed to both the first perturbation and the second perturbation;
generate, a third individual distribution density ratio metric from the third set of feature vectors; and
generate the latent variable separability measure by comparing the third individual distribution density ratio metric and the synthetic pairwise distribution representation.
